SEQUENCE LISTING (33 sequences)

SEQ ID NO: 1 - DNA, 2535 bp, Artificial Sequence
  cDNA sequence encoding pig T1R1
atgttgctctgggaggcgtgcctgctgggtctgcagctctccctctcctgctgctgggctttgggctgccagggcccagagtcctcccctgacttcagcctccctggggactaccttgtagctggcctcttccctctacacggtgaccacccagaggcgaggcgcagacccacagtggccttctgtgacagacccaacaccttcaacggccacggctaccacctcttccaagccatgcggtttggcatcgaggagataaacaactccagcgccctgctgcccaacgtcactctggggtatgagctatacgacgtgtgctcggagtccgcgaacgtgtacgccacgctgagcatcctgagcaaggctgggacaggctacgtggacatccaagaaaaccctgccctctattcccccaagaccgtggcggtcatcgggcccgacaccaccagccgcgctgccaccaccgcaggcctgctgggccccttcctggtgcccctggtcagctatgatgccagcagcacggtgctgggtgtgaagcgggtgtacccctctttcctgcgcaccatccccagcgatgaccaccaggtggaggtcttggtgctgctgctgcggaggttcaggtgggactggatctcggtggtgggcagcgaaggtgactacgggcagctgggggtgcaggcgctggaggaccaggcccgccggcagggcatctgcattgccttcaaggacatcgtgccgttctctgcccagccgggcgaccagaggatgcagagcatgatgggcagcccggctctcgcaaggaccaccgttgtggtcgttttctccggtcggcagctggccagggtgttctttgagtctgtggtgctggccaacctgacctccaaggtgtgggtcgcctccgaggactgggccatctccagacacatcagcagtgtgcccgggatccggggcatcggcacagtgctgggcgtggccatccccgggaggctggtgcccggcctgaaggagtttgaggaggcctacgtcagggcagagaaaggggcccttgggccttgccccaggggctcctggtgcagcaccaaccagctctgcacagactgctgggctttcacggcccaggagatgcccacacttggagcattctccatgagttctgcctacaacgtgtaccaggctgtctatgccgtggcccagggcctccaccagctcctgggctgtgcctctggcatctgttctcgaggccaagtctacccctggcagctcctggaggagatccgcaaggtcaatttccttctacgcaagcacactgtgacatttagtgacagtggggactcgctcagcggctatgacataattgcctgggactggagtggccccaagtggaccttcagggtcatcggctcgtcatcgtcgtcattgttgtcgccagttcagctagacataaatactaccaaaatcccatggcgtggcggggacaaccaggtgccgacgtccgtgtgttccagcgactgtcctgagggccaccagaaagtgatcatgagtttccaccactgctgctttgagtgtgtgccttgtgaggccgggaccttcctcaacgagagtgacccctacagctgccaaccctgtgggaaagaagagtgggcacctgagagaagccagacttgcttcccacgcaccgtggtattcttgacttggcatgagcccatctcttgggtgctgctagcagctaacacgatgttgctgctgctggtggctgggactgctggcctgtttgcttggtacctagacacccctgtggtgaggtccgctgggggcagaatgtgcttcctcatgctgggctccctggcagggggcagctgcggcctctatggcttttttggggagcccatgctgcccacatgcttgctgcgccaaggcctctttgcccttggttttgccatcttcctgtcctgcctgacaatccgctctttccaacttgtcttcatcttcaagttttctgccaagatgcccacattctttcacgcgtgggtccaaaaccatggtgctggcctgtttgtggtgatcagctcgataacccagctgcttatctgtctggtttggctggcggtgtggaccccactgcccaccagggaataccagcgcttcccacagctggtggtgcttgactgcacagaagcgaattcactgggcttaaggctggctttcacctacaacggtctcctctcggtcagcgcctttgcctgcagctacctgggtaaggacctgccagagaactacaatgaggccaaatgtgttaccttcagcctgctcctcaacttcgtgtcctggatcaccttcttcaccgtggccagtgtctaccagggcaagtacctggccgccgtcaacgtgtttgccatgctgagcagcctgagtggaggcttcagcggttatttcctccccaaatgctatatcatcctgtgccgcccagatctcaacagcacggagcacttccaggcctccatccaggactacagcaggcgctgcggctccacctga

SEQ ID NO: 2 - DNA, 2568 bp, Artificial Sequence
  cDNA sequence encoding pig T1R3
atgccaggcctgactctcctgggcctcacagctctcctgggcctcggggcgggggccccgctgtgcctgtcccggcagctcagcatgcaagggcactacgtgctgggggggctcttccccctgggctccaccgaggatgctgggctgggcgacaggacagagcccaacgccaccgtgtgtaccaggttctcgtccctcggcctgctctgggcactggccatgatgatggctgtggaggaaatcaacaacggatcggccctgctccctgggctgcgcctgggctacgacctcttcgacacctgctcagagcccatggtcgccatgaagcccagcctagtgttcatggccaaggctggcagccgcagcattgccacctactgcgactacacgcagtaccagccccgcgtgctggccgtcatcgggccccacatgtctgaggtcgccctggtcaccggcaagttcttcggcttcttcctcatgccgcaggtcagctacagcgccagcacggaccggctgagcaaccgcgagacgttcccctccttcttccgcacggtgcccagcgaccgcgtgcaggcagtggccatggtggagctgctgcaggagctgcgttggaactgggtggccgccgtgggcagcgacgacgagtacggccggcagggcctgagcctcttctccaacctggccaacgccaagggcatctgcatcgcgcacgagggcctggtgccgctgccccacgcccgtagcccaaggctggactgcacgcagggcctgctgccccaagtgaaccagagcagcgtgcaggtagtggtgctgttctcctccgcccgcgccgcccgcagcctcttcagccacagcctccactgcaggctctcgcccaaggtgtgggtggccagcgaggcctggctgacctcggacctggtcatgacactgccgggcatggaccaggtgggcaccgtgctcggcttcctgcaccagggtgccctgatgcccgagttctcggcctacgtgcagacccgcctggccctggccgccgaccccgcctactgcgcctcgctggctgccgagcggcaggggctggaggagcacgtggtggggcctcgctgcccccagtgtgactacgtcacattggagagcctgtccgaggggctgctgcaccaccggagcttccccgcctacgcggctgtgtacagcgtggcccaggcgctccacaacacgctgctctgcaacgcctcgggctgccccacgcgggagccggtgcagccctggcagctcctggagaacatgtacaacctgagcttccgtgcgcgcggcctgacactgcggttcgacaccagcgggaacgtggatatggattacgacctgaagctgtgggtgtggcaggacctgatgcccacactgcgcaccgtgggcactttcgacggccgcctgacgctccagcgtccccggatgcgctggcacacaacggggaacaaggagcccgtgtcccagtgctctcgacagtgcgaggagggccaggtgcgccgggtgaagggcttccactcctgctgctatgactgtgtggactgcaaggcgggcagctaccagcgcaacccagatgacaccctctgtgtccagtgtgaccaggaccagtggtccccggaccggagcacccggtgtttcccccgcaggcccaggttcctggcttggggggagccagccgtgctggtgctgctcgtgctgctgggcctggctctgggcctggtgctggcagccctggggctcttcatctggcaccgagacagcccactggttcaggcctccggtgggccacgggcctgcttcggcttggcctgcctgggcctcgtctgcctgagcgtcctcctgttcccgggccggcccggccctgccagctgcctggcccaacagccgctgctccacctccctctcactggctgcctgagcaccctcttcctgcaggcggctgagatctttgcgaggtcagagctgccgccaagctgggccgactggctctgtagccacctgtgggggccctgggcctggctggcggtgctgctcgccatgctggcacaagcagcgctctgcacctggtacctggtggccttcccaccggaggtggtgacagactggcgtgcgctgcccacagaggcactggtgcactgccgcgtgcactcctggatcagcttcagcctggtacatgctgccaatgccacgctggctttcctctgcttcctgggcaccttcctggtgcagagccagcctggctaccacaacagcgcccgcagcctgaccttcgccatgctggcctacttcatcacctgggtttcctttgtgcccctcctagccaacgtgcctgtggcctcccagcctgccgtgcacatgggtgccgccctcctctgtgtgctgggcatcctggccactttccacctgcccaagtgctacctgctgctgaggtggccagagctcaacacccccacgttcttcctggggggatgccagagggcaggggggcggggaggagactctgggaaaaaacaaatgacccctgacccagtgacctccccccagtga

SEQ ID NO: 3 - DNA, 19 bp, Artificial Sequence
  synthetic T1R3 GSP forward gene specific primer for amplifying T1R3
actgccgcgtgcactcctg

SEQ ID NO: 4 - DNA, 19 bp, Artificial Sequence
  synthetic T1R3 GSP reverse gene specific primer sequence for amplifying T1R3
ggaggccacaggcacgttg

SEQ ID NO: 5 - DNA, 31 bp, Artificial Sequence
  synthetic T1R1 DP forward degenerate primer sequence for amplifying T1R1
ggcagtacccctccttcctgmgnacnathcc

SEQ ID NO: 6 - DNA, 25 bp, Artificial Sequence
  synthetic T1R1 DP reverse degenerate primer for amplifying T1R1
cctgcacgcccagctgnccrtartc

SEQ ID NO: 7 - DNA, 21 bp, Artificial Sequence
  synthetic forward primer sequence for amplifying T1R3
atgccaggcctgactctcctg

SEQ ID NO: 8 - DNA, 21 bp, Artificial Sequence
  synthetic reverse primer sequence for amplifying T1R3
tcactgtggggtcaagggtgg

SEQ ID NO: 9 - DNA, 20 bp, Artificial Sequence
  synthetic reverse nested primer sequence for amplifying T1R3
tcagggcacagcctgctggt

SEQ ID NO: 10 - DNA, 18 bp, Artificial Sequence
  synthetic forward primer sequence for amplifying T1R1
atgccaggctcagggcgg

SEQ ID NO: 11 - DNA, 20 bp, Artificial Sequence
  synthetic forward nested primer sequence for amplifying T1R1
atgttgctctgggaggcgtg

SEQ ID NO: 12 - DNA, 18 bp, Artificial Sequence
  synthetic reverse primer sequence for amplifying T1R1
tcaggtggagccgcagcg

SEQ ID NO: 13 - protein, 844 aa, Sus scrofa
  pT1R1 amino acid sequence
MLLWEACLLGLQLSLSCCWALGCQGPESSPDFSLPGDYLVAGLFPLHGDHPEARRRPTVAFCDRPNTFNGHGYHLFQAMRFGIEEINNSSALLPNVTLGYELYDVCSESANVYATLSILSKAGTGYVDIQENPALYSPKTVAVIGPDTTSRAATTAGLLGPFLVPLVSYDASSTVLGVKRVYPSFLRTIPSDDHQVEVLVLLLRRFRWDWISVVGSEGDYGQLGVQALEDQARRQGICIAFKDIVPFSAQPGDQRMQSMMGSPALARTTVVVVFSGRQLARVFFESVVLANLTSKVWVASEDWAISRHISSVPGIRGIGTVLGVAIPGRLVPGLKEFEEAYVRAEKGALGPCPRGSWCSTNQLCTDCWAFTAQEMPTLGAFSMSSAYNVYQAVYAVAQGLHQLLGCASGICSRGQVYPWQLLEEIRKVNFLLRKHTVTFSDSGDSLSGYDIIAWDWSGPKWTFRVIGSSSSSLLSPVQLDINTTKIPWRGGDNQVPTSVCSSDCPEGHQKVIMSFHHCCFECVPCEAGTFLNESDPYSCQPCGKEEWAPERSQTCFPRTVVFLTWHEPISWVLLAANTMLLLLVAGTAGLFAWYLDTPVVRSAGGRMCFLMLGSLAGGSCGLYGFFGEPMLPTCLLRQGLFALGFAIFLSCLTIRSFQLVFIFKFSAKMPTFFHAWVQNHGAGLFVVISSITQLLICLVWLAVWTPLPTREYQRFPQLVVLDCTEANSLGLRLAFTYNGLLSVSAFACSYLGKDLPENYNEAKCVTFSLLLNFVSWITFFTVASVYQGKYLAAVNVFAMLSSLSGGFSGYFLPKCYIILCRPDLNSTEHFQASIQDYSRRCGST

SEQ ID NO: 14 - protein, 855 aa, Sus scrofa
  pT1R3 amino acid sequence
MPGLTLLGLTALLGLGAGAPLCLSRQLSMQGHYVLGGLFPLGSTEDAGLGDRTEPNATVCTRFSSLGLLWALAMMMAVEEINNGSALLPGLRLGYDLFDTCSEPMVAMKPSLVFMAKAGSRSIATYCDYTQYQPRVLAVIGPHMSEVALVTGKFFGFFLMPQVSYSASTDRLSNRETFPSFFRTVPSDRVQAVAMVELLQELRWNWVAAVGSDDEYGRQGLSLFSNLANAKGICIAHEGLVPLPHARSPRLDCTQGLLPQVNQSSVQVVVLFSSARAARSLFSHSLHCRLSPKVWVASEAWLTSDLVMTLPGMDQVGTVLGFLHQGALMPEFSAYVQTRLALAADPAYCASLAAERQGLEEHVVGPRCPQCDYVTLESLSEGLLHHRSFPAYAAVYSVAQALHNTLLCNASGCPTREPVQPWQLLENMYNLSFRARGLTLRFDTSGNVDMDYDLKLWVWQDLMPTLRTVGTFDGRLTLQRPRMRWHTTGNKEPVSQCSRQCEEGQVRRVKGFHSCCYDCVDCKAGSYQRNPDDTLCVQCDQDQWSPDRSTRCFPRRPRFLAWGEPAVLVLLVLLGLALGLVLAALGLFIWHRDSPLVQASGGPRACFGLACLGLVCLSVLLFPGRPGPASCLAQQPLLHLPLTGCLSTLFLQAAEIFARSELPPSWADWLCSHLWGPWAWLAVLLAMLAQAALCTWYLVAFPPEVVTDWRALPTEALVHCRVHSWISFSLVHAANATLAFLCFLGTFLVQSQPGYHNSARSLTFAMLAYFITWVSFVPLLANVPVASQPAVHMGAALLCVLGILATFHLPKCYLLLRWPELNTPTFFLGGCQRAGGRGGDSGKKQMTPDPVTSPQ

SEQ ID NO: 15 - protein, 844 aa, Sus scrofa; identical to SEQ ID NO: 13
  porcine T1R1 amino acid sequence

SEQ ID NO: 16 - protein, 841 aa, Felis catus
  feline T1R1 amino acid sequence
MSLPAAHLVGLQLSLSCCWALSCHSTETSADFSLPGDYLLAGLFPLHSDCPGVRHRPTVTLCDRPDSFNGHGYHLFQAMRFGIEEINNSTALLPNVTLGYQLYDVCSESANVYATLNVLSLLGTHHVEIRADPSHYSPAALAVIGPDTTNHAATTAALLSPFLVPLISYEASSVTLGVKRHYPSFLRTIPSDKHQVEAMVLLLQSFGWVWISVVGSDGDYGQLGVQALEEQATQQGICVAFKDIIPFSARPGDERMQSIMHHLARARTTVVVVFSSRQLARVFFESVVLANLTAKVWIASEDWAISRHISNVPGIQGIGTVLGVAIQQRLVPGLKEFEEAYVQADKGAPGPCSRTSECSSNQLCRECRAFTAEQMPTLGAFSMSSAYNAYRAVYAVAHGLHQLLGCASGACSRDRVYPWQLLEQIRKVNFLLHKDTVRFNDNGDPLSGYDIIAWDWSGPKWNFRVIGSSMWPPVQLDINKTKIRWHGKDNQVPKSVCSSDCLEGHQRVISGFYHCCFECVPCEAGSFLNKSDLHSCQPCGKEKWAPAGSETCFPRTVVFLTWHETISWVLLAANTLLLLLVTGTAGLFAWHLDTPVVKSAGGRLCFFMLGSLAGGSCGLYGFFGEPTLPTCLLRQSLLALGFAIFLSCLTIRSFQLVFIFKFSAKVPTFYRAWVQNHGPGLFVVISSMAQLLICLTWLAVWTPLPTREYQRFPQLVVLDCTEANSPGFMLAFAYNGLLSVSAFACSYLGKDLPENYNEAKCVTFSLLLNFVSWIAFFTTASVYQGKYLPAVNVLAALSSLSGGFSGYFLPKCYVILCRPKFNSTQHFQASIQEYTRRCGST

SEQ ID NO: 17 - protein, 840 aa, Homo sapiens
  human T1R1 amino acid sequence
MLLCTARLVGLQLLISCCWAFACHSTESSPDFTLPGDYLLAGLFPLHSGCLQVRHRPEVTLCDRSCSFNEHGYHLFQAMRLGVEEINNSTALLPNITLGYQLYDVCSDSANVYATLRVLSLPGQHHIELQGDLLHYSPTVLAVIGPDSTNRAATTAALLSPFLVPMISYAASSETLSVKRQYPSFLRTIPNDKYQVETMVLLLQKFGWTWISLVGSSDDYGQLGVQALENQATGQGICIAFKDIMPFSAQVGDERMQCLMRHLAQAGATVVVVFSSRQLARVFFESVVLTNLTGKVWVASEAWALSRHITGVPGIQRIGMVLGVAIQKRAVPGLKAFEEAYARADKKAPRPCHKGSWCSSNQLCRECQAFMAHTMPKLKAFSMSSAYNAYRAVYAVAHGLHQLLGCASGACSRGRVYPWQLLEQIHKVHFLLHKDTVAFNDNRDPLSSYNIIAWDWNGPKWTFTVLGSSTWSPVQLNINETKIQWHGKDNQVPKSVCSSDCLEGHQRVVTGFHHCCFECVPCGAGTFLNKSDLYRCQPCGKEEWAPEGSQTCFPRTVVFLALREHTSWVLLAANTLLLLLLLGTAGLFAWHLDTPVVRSAGGRLCFLMLGSLAAGSGSLYGFFGEPTRPACLLRQALFALGFTIFLSCLTVRSFQLIIIFKFSTKVPTFYHAWVQNHGAGLFVMISSAAQLLICLTWLVVWTPLPAREYQRFPHLVMLECTETNSLGFILAFLYNGLLSISAFACSYLGKDLPENYNEAKCVTFSLLFNFVSWIAFFTTASVYDGKYLPAANMMAGLSSLSSGFGGYFLPKCYVILCRPDLNSTEHFQASIQDYTRRCGS

SEQ ID NO: 18 - protein, 842 aa, Mus musculus
  mouse T1R1 amino acid sequence
MLFWAAHLLLSLQLAVAYCWAFSCQRTESSPGFSLPGDFLLAGLFSLHADCLQVRHRPLVTSCDRSDSFNGHGYHLFQAMRFTVEEINNSTALLPNITLGYELYDVCSESSNVYATLRVLAQQGTGHLEMQRDLRNHSSKVVALIGPDNTDHAVTTAALLSPFLMPLVSYEASSVILSGKRKFPSFLRTIPSDKYQVEVIVRLLQSFGWVWISLVGSYGDYGQLGVQALEELATPRGICVAFKDVVPLSAQAGDPRMQRMMLRLARARTTVVVVFSNRHLAGVFFRSVVLANLTGKVWIASEDWAISTYITNVPGIQGIGTVLGVAIQQRQVPGLKEFEESYVQAVTGAPRTCPEGSWCGTNQLCRECHAFTTWNMPELGAFSMSAAYNVYEAVYAVAHGLHQLLGCTSGTCARGPVYPWQLLQQIYKVNFLLHKKTVAFDDNGDPLGYYDIIAWDWNGPEWTFEVIGSASLSPVHLDINKTKIQWHGKNNQVPVSVCTRDCLEGHHRLVMGSHHCCFECMPCEAGTFLNTSELHTCQPCGTEEWAPEGSSACFSRTVEFLGWHEPISLVLLAANTLLLLLLIGTAGLFAWRLHTPVVRSAGGRLCFLMLGSLVAGSCSLYSFFGEPTVPACLLRQPLFSLGFAIFLSCLTIRSFQLVIIFKFSTKVPTFYHTWAQNHGAGIFVIVSSTVHLFLCLTWLAMWTPRPTREYQRFPHLVILECTEVNSVGFLVAFAHNILLSISTFVCSYLGKELPENYNEAKCVTFSLLLHFVSWIAFFTMSSIYQGSYLPAVNVLAGLATLSGGFSGYFLPKCYVILCRPELNNTEHFQASIQDYTRRCGTT

SEQ ID NO: 19 - protein, 840 aa, Rattus norvegicus
  rat T1R1 amino acid sequence
MLFWAAHLLLSLQLVYCWAFSCQRTESSPGFSLPGDFLLAGLFSLHGDCLQVRHRPLVTSCDRPDSFNGHGYHLFQAMRFTVEEINNSSALLPNITLGYELYDVCSESANVYATLRVLALQGPRHIEIQKDLRNHSSKVVAFIGPDNTDHAVTTAALLGPFLMPLVSYEASSVVLSAKRKFPSFLRTVPSDRHQVEVMVQLLQSFGWVWISLIGSYGDYGQLGVQALEELAVPRGICVAFKDIVPFSARVGDPRMQSMMQHLAQARTTVVVVFSNRHLARVFFRSVVLANLTGKVWVASEDWAISTYITSVTGIQGIGTVLGVAVQQRQVPGLKEFEESYVRAVTAAPSACPEGSWCSTNQLCRECHTFTTRNMPTLGAFSMSAAYRVYEAVYAVAHGLHQLLGCTSEICSRGPVYPWQLLQQIYKVNFLLHENTVAFDDNGDTLGYYDIIAWDWNGPEWTFEIIGSASLSPVHLDINKTKIQWHGKNNQVPVSVCTTDCLAGHHRVVVGSHHCCFECVPCEAGTFLNMSELHICQPCGTEEWAPKESTTCFPRTVEFLAWHEPISLVLIAANTLLLLLLVGTAGLFAWHFHTPVVRSAGGRLCFLMLGSLVAGSCSFYSFFGEPTVPACLLRQPLFSLGFAIFLSCLTIRSFQLVIIFKFSTKVPTFYRTWAQNHGAGLFVIVSSTVHLLICLTWLVMWTPRPTREYQRFPHLVILECTEVNSVGFLLAFTHNILLSISTFVCSYLGKELPENYNEAKCVTFSLLLNFVSWIAFFTMASIYQGSYLPAVNVLAGLTTLSGGFSGYFLPKCYVILCRPELNNTEHFQASIQDYTRRCGTT

SEQ ID NO: 20 - protein, 855 aa, Sus scrofa
  porcine T1R3 amino acid sequence
MPGLTLLGLTALLGLGAGAPLCLSRQLSMQGHYVLGGLFPLGSTEDAGLGDRTEPNATVCTRFSSLGLLWALAMMMAVEEINNGSALLPGLRLGYDLFDTCSEPMVAMKPSLVFMAKAGSRSIATYCDYTQYQPRVLAVIGPHMSEVALVTGKFFGFFLMPQVSYSASTDRLSNRETFPSFFRTVPSDRVQAVAMVELLQELRWNWVAAVGSDDEYGRQGLSLFSNLANAKGICIAHEGLVPLPHARSPRLDCTQGLLPQVNQSSVQVVVLFSSARAARSLFSHSLHCRLSPKVWVASEAWLTSDLVMTLPGMDQVGTVLGFLHQGALMPEFSAYVQTRLALAADPAYCASLAAERQGLEEHVVGPRCPQCDYVTLESLSGGLLHHRSFPAYAAVYSVAQALHNTLLCNASGCPTREPVQPWQLLENMYNLSFRARGLTLRFDTSGNVDMDYDLKLWVWQDLMPTLRTVGTFDGRLTLQRPRMRWHTTGNKEPVSQCSRQCEEGQVRRVKGFHSCCYDCVDCKAGSYQRNPDDTLCVQCDQDQWSPDRSTRCFPRRPRFLAWGEPAVLVLLVLLGLALGLVLAALGLFIWHRDSPLVQASGGPRACFGLACLGLVCLSVLLFPGRPGPASCLAQQPLLHLPLTGCLSTLFLQAAEIFARSELPPSWADWLCSHLWGPWAWLAVLLAMLAQAALCTWYLVAFPPEVVTDWRALPTEALVHCRVHSWISFSLVHAANATLAFLCFLGTFLVQSQPGYHNSARSLTFAMLAYFITWVSFVPLLANVPVASQPAVHMGAALLCVLGILATFHLPKCYLLLRWPELNTPTFFLGGCQRAGGRGGDSGKKQMTPDPVTSPQ

SEQ ID NO: 21 - protein, 865 aa, Felis catus
  feline T1R3 amino acid sequence
MPGLALLGLTALLGLTALLDHGEGATSCLSQQLRMQGDYVLGGLFPLGSAEGTGLGDGLQPNATVCTRFSSLGLLWALAVKMAVEEINNGSALLPGLHLGYDLFDTCSEPMVAMKPSLVFMAKAGSCSIAAYCNYTQYQPRVLAVIGPHSSELALVTGKFFSFFLVPQVSYGASTDRLSNREIFPSFFRTVPSDQVQVAAMVELLEELGWNWVAAVGSDDEYGRQGLSLFSGLASARGICIAHEGLVPLPPGSLRLGALQGLLRQVNQSSVQVVVLFSSAHAARTLFSYSIRCKLSPKVWVASEAWLTSDLVMTLPGMPGVGTVLGFLQQGAPMPEFPSYVRTRLALAADPAFCASLDAEQPGLEEHVVGPRCPQCDHVTLENLSAGLLHHQTFAAYAAVYGVAQALHNTLRCNASGCPRREPVRPWQLLENMYNVSFRARGLALQFDASGNVNVDYDLKLWVWQDPTPELRTVGTFKGRLELWRSQMCWHTPGKQQPVSQCSRQCKEGQVRRVKGFHSCCYNCVDCKAGSYQRNPDDLLCTQCDQDQWSPDRSTRCFARKPMFLAWGEPAVLLLLALLALALGLALAALGLFLWHSDSPLVQASGGPRACFGLACLGLVCLSVLLFPGQPGPASCLAQQPLFHLPLTGCLSTFFLQAAEIFVGSELPPSWAEKMRGRLRGPWAWLVVLLAMLAEAALCAWYLVAFPPEVVTDWRVLPTEALVHCHVHSWISFGLVHATNAMLAFLCFLGTFLVQSRPGRYNGARGLTFAMLAYFITWISFVPLFANVHVAYQPAVQMGTILLCALGILATFHLPKCYLLLQRPELNTPEFFLEDNARAQGSSWGQGRGESGQKQVTPDPVTSPQ

SEQ ID NO: 22 - protein, 852 aa, Homo sapiens
  human T1R3 amino acid sequence
MLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELLQEFGWNWVAALGSDDEYGRQGLSIFSALAAARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

SEQ ID NO: 23 - protein, 858 aa, Mus musculus
  mouse T1R3 amino acid sequence
MPALAIMGLSLAAFLELGMGASLCLSQQFKAQGDYILGGLFPLGSTEEATLNQRTQPNSILCNRFSPLGLFLAMAMKMAVEEINNGSALLPGLRLGYDLFDTCSEPVVTMKSSLMFLAKVGSQSIAAYCNYTQYQPRVLAVIGPHSSELALITGKFFSFFLMPQVSYSASMDRLSDRETFPSFFRTVPSDRVQLQAVVTLLQNFSWNWVAALGSDDDYGREGLSIFSSLANARGICIAHEGLVPQHDTSGQQLGKVLDVLCQVNQSKVQVVVLFASARAVYSLFSYSIHHGLSPKVWVASESWLTSDLVMTLPNIARVGTVLGFLQRGALLPEFSHYVETHLALAADPAFCASLNAELDLEEHVMGQRCPQCDDIMLQNLSSGLLQNLSAGQLHHQIFATYAAVYSVAQALHNTLQCNVSHCHVSEHVLPWQLLENMYNMSFHARDLTLQFDAEGNVDMEYDLKMWVWQSPTPVLHTVGTFNGTLQLQQSKMYWPGNQVPVSQCSRQCKDGQVRRVKGFHSCCYDCVDCKAGSYRKHPDDFTCTPCNQDQWSPEKSTACLPRRPKFLAWGEPVVLSLLLLLCLVLGLALAALGLSVHHWDSPLVQASGGSQFCFGLICLGLFCLSVLLFPGRPSSASCLAQQPMAHLPLTGCLSTLFLQAAETFVESELPLSWANWLCSYLRGLWAWLVVLSATFVEAALCAWYLTAFPPEVVTDWSVLPTEVLEHCHVRSWVSLGLVHITNAMLAFLCFLGTFLVQSQPGRYNRARGLTFAMLAYFITWVSFVPLLANVQVAYQPAVQMGAILVCALGILVTFHLPKCYVLLWLPKLNTQEFFLGRNAKKAADENSGGGEAAQEHNE

SEQ ID NO: 24 - protein, 858 aa, Rattus norvegicus
  rat T1R3 amino acid sequence
MPGLAILGLSLAAFLELGMGSSLCLSQQFKAQGDYILGGLFPLGTTEEATLNQRTQPNGILCTRFSPLGLFLAMAMKMAVEEINNGSALLPGLRLGYDLFDTCSEPVVTMKPSLMFMAKVGSQSIAAYCNYTQYQPRVLAVIGPHSSELALITGKFFSFFLMPQVSYSASMDRLSDRETFPSFFRTVPSDRVQLQAVVTLLQNFSWNWVAALGSDDDYGREGLSIFSGLANSRGICIAHEGLVPQHDTSGQQLGKVVDVLRQVNQSKVQVVVLFASARAVYSLFSYSILHDLSPKVWVASESWLTSDLVMTLPNIARVGTVLGFLQRGALLPEFSHYVETRLALAADPTFCASLKAELDLEERVMGPRCSQCDYIMLQNLSSGLMQNLSAGQLHHQIFATYAAVYSVAQALHNTLQCNVSHCHTSEPVQPWQLLENMYNMSFRARDLTLQFDAKGSVDMEYDLKMWVWQSPTPVLHTVGTFNGTLQLQHSKMYWPGNQVPVSQCSRQCKDGQVRRVKGFHSCCYDCVDCKAGSYRKHPDDFTCTPCGKDQWSPEKSTTCLPRRPKFLAWGEPAVLSLLLLLCLVLGLTLAALGLFVHYWDSPLVQASGGSLFCFGLICLGLFCLSVLLFPGRPRSASCLAQQPMAHLPLTGCLSTLFLQAAEIFVESELPLSWANWLCSYLRGPWAWLVVLLATLVEAALCAWYLMAFPPEVVTDWQVLPTEVLEHCRMRSWVSLGLVHITNAVLAFLCFLGTFLVQSQPGRYNRARGLTFAMLAYFIIWVSFVPLLANVQVAYQPAVQMGAILFCALGILATFHLPKCYVLLWLPELNTQEFFLGRSPKEASDGNSGSSEATRGHSE

SEQ ID NO: 25 - protein, 844 aa, Sus scrofa; identical to SEQ ID NO: 13
  pig T1R1 amino acid sequence

SEQ ID NO: 26 - protein, 852 aa, Homo sapiens; identical to SEQ ID NO: 22
  human T1R3 amino acid sequence

SEQ ID NO: 27 - protein, 858 aa, Mus musculus
  mouse T1R3 amino acid sequence
MPALAIMGLSLAAFLELGMGASLCLSQQFKAQGDYILGGLFPLGSTEEATLNQRTQPNSIPCNRFSPLGLFLAMAMKMAVEEINNGSALLPGLRLGYDLFDTCSEPVVTMKSSLMFLAKVGSQSIAAYCNYTQYQPRVLAVIGPHSSELALITGKFFSFFLMPQVSYSASMDRLSDRETFPSFFRTVPSDRVQLQAVVTLLQNFSWNWVAALGSDDDYGREGLSIFSSLANARGICIAHEGLVPQHDTSGQQLGKVLDVLRQVNQSKVQVVVLFASARAVYSLFSYSIHHGLSPKVWVASESWLTSDLVMTLPNIARVGTVLGFLQRGALLPEFSHYVETHLALAADPAFCASLNAELDLEEHVMGQRCPRCDDIMLQNLSSGLLQNLSAGQLHHQIFATYAAVYSVAQALHNTLQCNVSHCHVSEHVLPWQLLENMYNMSFHARDLTLQFDAEGNVDMEYDLKMWVWQSPTPVLHTVGTFNGTLQLQQSKMYWPGNQVPVSQCSRQCKDGQVRRVKGFHSCCYDCVDCKAGSYRKHPDDFTCTPCNQDQWSPEKSTACLPRRPKFLAWGEPVVLSLLLLLCLVLGLALAALGLSVHHWDSPLVQASGGSQFCFGLICLGLFCLSVLLFPGRPSSASCLAQQPMAHLPLTGCLSTLFLQAAETFVESELPLSWANWLCSYLRGLWAWLVVLLATFVEAALCAWYLIAFPPEVVTDWSVLPTEVLEHCHVRSWVSLGLVHITNAMLAFLCFLGTFLVQSQPGRYNRARGLTFAMLAYFITWVSFVPLLANVQVAYQPAVQMGAILVCALGILVTFHLPKCYVLLWLPKLNTQEFFLGRNAKKAADENSGGGEAAQGHNE

SEQ ID NO: 28 - protein, 855 aa, Sus scrofa; identical to SEQ ID NO: 14
  pig T1R3 amino acid sequence

SEQ ID NO: 29 - protein, 855 aa, Sus scrofa; identical to SEQ ID NO: 14
  pig T1R3 amino acid sequence

SEQ ID NO: 30 - protein, 245 aa, Artificial Sequence
  glutathione S-transferase (GST) amino acid sequence
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPNSRVDSSGRIVTD

SEQ ID NO: 31 - protein, 121 aa, Artificial Sequence
  LacZ amino acid sequence
MTMITPSAQLTLTKGNKSWSSTAVAAALELVDPPGCRNSISSLSIPSTSRGGPVPNSPYSESYYARSLAVVLQRRDWENPGVTQLNRLAAHPPFASWRNSEEARTDRPSQQLRSLNGEWKL

SEQ ID NO: 32 - protein, 10 aa, Artificial Sequence
  c-myc amino acid sequence
EQKLISEEDL

SEQ ID NO: 33 - protein, 200 aa, Artificial Sequence
  poly gly sequence
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

(19) United States
(12) Patent Application Publication
(10) Pub. No.: US 2011/0082220 A1
(43) Pub. Date: Apr. 7, 2011

(54) PORCINE UMAMI TASTE RECEPTORS AND USES THEREFOR

(75) Inventors: Kirk Klasing, Davis, CA (US); Roseline Holt, Woodland, CA (US); Eugeni Roura, Barcelona (ES)

(73) Assignees: The Regents of The University of California, Oakland, CA (US); LUCTA, Montornes del Valles (ES)

(21) Appl. No.: 12/900,343

(22) Filed: Oct. 7, 2010

Related U.S. Application Data
(62) Division of application No. 12/181,742, filed on Jul. 29, 2008.
(60) Provisional application No. 60/997,644, filed on Oct. 3, 2007.

(51) Int. Cl. A61K 47/16; G01N 33/566; A23L 1/22

(52) U.S. Cl. 514/773; 435/7.21; 426/650; 514/784

(57) ABSTRACT

The present invention provides nucleic acids encoding porcine taste receptors, polypeptides encoded by the nucleic acids, and methods of using the nucleic acids and polypeptides to identify compounds that enhance umami taste.

### CROSS-REFERENCE TO RELATED APPLICATIONS

This application is a divisional of U.S. application Ser. No. 12/181,742, filed on Jul. 29, 2008, which claims the benefit of U.S. Provisional Application No. 60/997,644, filed on Oct. 3, 2007, the entire disclosures of which is hereby incorporated herein by reference for all purposes.

### BACKGROUND OF THE INVENTION

The sense of taste gives animals the ability to evaluate what they eat and drink. At a basic level, the sense of taste and the resulting ability to evaluate what is ingested, ensures promoting the ingestion of nutritious substances while preventing the consumption of potentially harmful substances. Additionally, humans and other animals develop taste preferences thus choosing certain foods over others.

To date, five major taste sensations have been described: sweet, sour, salty, bitter and umami. The majority of research has focused on characterizing the receptors responsible for the various taste sensations in humans.

The human umami receptor has two polypeptide chains, each coded by a different gene. When the receptor composed of the two peptides binds its ligand, a series of signal transduction events is initiated resulting in the release of calcium into the cytoplasm. The calcium flux, in turn, triggers an action potential at the cell membrane of the taste cell. The taste cell synapses with efferent neurons that communicate the taste to the brain. No information on a pig umami receptor has been available and there is limited information on the ability of pigs to taste umami flavor.

Compositions and methods for increasing the palatability of feed, feed supplements, and medicaments to pigs and for identifying compounds that increase the palatability of such substances to pigs are needed in the art. The present invention addresses these and other needs.

### BRIEF SUMMARY OF THE INVENTION

The invention provides nucleic acids encoding pig taste receptors, polypeptides encoded by the nucleic acids and methods for using the nucleic acids and polypeptides.

One embodiment of the invention provides an isolated taste receptor, comprising a first taste receptor polypeptide selected from the group consisting of: a pT1R1 polypeptide encoded by a nucleotide sequence that hybridizes under highly stringent hybridization conditions to a nucleotide sequence of SEQ ID NO:1 or a complement thereof; and a pT1R3 polypeptide encoded by a nucleotide sequence that hybridizes under highly stringent hybridization conditions to a nucleotide sequence of SEQ ID NO:2 or a complement thereof. In some embodiments, the taste receptor further comprises a second taste receptor polypeptide, wherein the first taste receptor polypeptide and the second taste receptor polypeptide are independently selected from the group consisting of: a pT1R1 polypeptide (SEQ ID NO:13) encoded by a nucleotide sequence that hybridizes under highly stringent hybridization conditions to a nucleotide sequence of SEQ ID NO:1 or a complement thereof; a pT1R3 polypeptide (SEQ ID NO:14) encoded by a nucleotide sequence that hybridizes under highly stringent hybridization conditions to a nucleotide sequence of SEQ ID NO:2 or a complement thereof.

In some embodiments, the isolated taste receptor comprises a first taste receptor polypeptide selected from the group consisting of: a pT1R1 polypeptide encoded by a nucleotide sequence sharing at least about 95%, 96%, 97%, 98% or 99% sequence identity to SEQ ID NO:1 and a pT1R3 polypeptide encoded by a nucleotide sequence sharing at least about 95%, 96%, 97%, 98% or 99% sequence identity to SEQ ID NO:2. In some embodiments, the taste receptor further comprises a second taste receptor polypeptide, wherein the first taste receptor polypeptide and the second taste receptor polypeptide are independently selected from the group consisting of: a pT1R1 polypeptide encoded by a nucleotide sequence sharing at least about 95%, 96%, 97%, 98% or 99% sequence identity to SEQ ID NO:1 and a pT1R3 polypeptide encoded by a nucleotide sequence sharing at least about 95%, 96%, 97%, 98% or 99% sequence identity to SEQ ID NO:2.

The first and second taste receptor polypeptides can both be a T1R1 polypeptide or can both be a T1R3 polypeptide. In some embodiments, the first taste receptor polypeptide is a pT1R1 polypeptide encoded by a nucleotide sequence that hybridizes under highly stringent hybridization conditions to a nucleotide sequence of SEQ ID NO:1 or a complement thereof; and the second taste receptor polypeptide is a pT1R3 polypeptide encoded by a nucleotide sequence that hybridizes under highly stringent hybridization conditions to a nucleotide sequence of SEQ ID NO:2 or a complement thereof. In some embodiments, the first taste receptor polypeptide is a pT1R1 polypeptide encoded by a nucleotide sequence sharing at least about 95%, 96%, 97%, 98% or 99% sequence identity to SEQ ID NO:1; and the second taste receptor polypeptide is a pT1R3 polypeptide encoded by a nucleotide sequence sharing at least about 95%, 96%, 97%, 98% or 99% sequence identity to SEQ ID NO:2.

In some embodiments, the isolated pT1R1 is encoded by a nucleic acid amplified from porcine genomic DNA, mRNA or cDNA template using a forward primer selected from the group consisting of SEQ ID NO:5, SEQ ID NO:10 and SEQ ID NO:11, and a reverse primer selected from the group consisting of SEQ ID NO:6 and SEQ ID NO:12. In some embodiments, the isolated pT1R3 is encoded by a nucleic acid amplified from porcine genomic DNA, mRNA or cDNA template using a forward primer selected from the group consisting of SEQ ID NO:3, SEQ ID NO:7, and a reverse primer selected from the group consisting of SEQ ID NO:4, SEQ ID NO:8 and SEQ ID NO:9.

In some embodiments, the isolated taste receptor is a porcine taste receptor. In some embodiments, the receptor has an activity selected from the group consisting of: G protein coupled receptor activity; protein kinase activity; and cyclic AMP elevation activity. In some embodiments, the first taste receptor polypeptide and the second taste receptor polypeptide are non-covalently linked. In some embodiments, the first taste receptor polypeptide and the second taste receptor polypeptide are covalently linked. In some embodiments, the receptor binds to umami taste ligands (e.g., an umami taste ligand selected from the group consisting of: glutamine and glutamate). In some embodiments, the first taste receptor polypeptide and the second taste receptor polypeptide are recombinant.

The invention also provides host cells (e.g., bacterial, mammalian, and yeast cells) comprising the isolated taste receptors. In some embodiments, the host cell does not express pT1R1 or pT1R3.

A further aspect of the invention provides methods of identifying a compound that modulates (e.g., increases or decreases) taste signal transduction in taste cells, the method comprising the steps of

(i) contacting the compound with a taste receptor comprising a first taste receptor polypeptide selected from the group consisting of: a pT1R1 polypeptide encoded by a nucleotide sequence that hybridizes under highly stringent hybridization conditions to a nucleotide sequence of SEQ ID NO:1 or a complement thereof; and a pT1R3 polypeptide encoded by a nucleotide sequence that hybridizes under highly stringent hybridization conditions to a nucleotide sequence of SEQ ID NO:2 or a complement thereof; and

(ii) determining a physical or functional effect of the compound upon the receptor, thereby identifying a compound that modulates taste signal transduction.

A related aspect of the invention provides methods of identifying a compound that modulates (e.g., increases or decreases) taste signal transduction in taste cells, the method comprising the steps of

(i) contacting the compound with a taste receptor comprising a first taste receptor polypeptide selected from the group consisting of: a pT1R1 polypeptide encoded by a nucleotide sequence sharing at least about 95%, 96%, 97%, 98% or 99% sequence identity to SEQ ID NO:1 and a pT1R3 polypeptide encoded by a nucleotide sequence sharing at least about 95%, 96%, 97%, 98% or 99% sequence identity to SEQ ID NO:2; and

(ii) determining the functional effect of the compound upon the receptor, thereby identifying a compound that modulates taste signal transduction.

With respect to the embodiments of the methods, in some embodiments, the taste receptor further comprises a second taste receptor polypeptide, wherein the first taste receptor polypeptide and the second taste receptor polypeptide are independently selected from the group consisting of: a pT1R1 polypeptide encoded by a nucleotide sequence that hybridizes under highly stringent hybridization conditions to a nucleotide sequence of SEQ ID NO:1 or a complement thereof; and a pT1R3 polypeptide encoded by a nucleotide sequence that hybridizes under highly stringent hybridization conditions to a nucleotide sequence of SEQ ID NO:2 or a complement thereof. In some embodiments, the first taste receptor polypeptide is a pT1R1 polypeptide encoded by a nucleotide sequence sharing at least about 95%, 96%, 97%, 98% or 99% sequence identity to SEQ ID NO:1; and the second taste receptor polypeptide is a pT1R3 polypeptide encoded by a nucleotide sequence sharing at least about 95%, 96%, 97%, 98% or 99% sequence identity to SEQ ID NO:2.

The first and second taste receptor polypeptides can be the same or different subtypes. The first and second taste receptor polypeptides can both be a T1R1 polypeptide or can both be a T1R3 polypeptide. In some embodiments, the first taste receptor polypeptide is a pT1R1 polypeptide encoded by a nucleotide sequence that hybridizes under highly stringent hybridization conditions to a nucleotide sequence of SEQ ID NO:1 or a complement thereof; and the second taste receptor polypeptide is a pT1R3 polypeptide is encoded by a nucleotide sequence that hybridizes under highly stringent hybridization conditions to a nucleotide sequence of SEQ ID NO:2 or a complement thereof. Further embodiments of the receptors, as described herein, can also apply to the methods.

In some embodiments, the first taste receptor polypeptide and the second taste receptor polypeptide are non-covalently linked. In some embodiments, the first taste receptor polypeptide and the second taste receptor polypeptide are covalently linked. In some embodiments, the receptor is recombinant.

In some embodiments, the receptor has an activity selected from the group consisting of: G protein coupled receptor activity; protein kinase activity; cyclic AMP elevation activity, and combinations thereof. In some embodiments, the functional effect is measured in vitro. In some embodiments, the functional effect is measured in vivo. In some embodiments, the functional effect is measured ex vivo. In some embodiments, the functional effect is receptor-mediated calcium flux, e.g., from the extracellular space or from intracellular calcium stores.

In some embodiments, the receptor is expressed in a cell that does not express pT1R1, pT1R2, or pT1R3. In some embodiments, the receptor is expressed in a cell that does not express pT1R1. In some embodiments, the receptor is expressed in a cell that does not express pT1R3. In some embodiments, the cell is a mammalian cell, a bacterial cell, or a yeast cell.

A further aspect of the invention provides an isolated nucleic acid comprising a nucleic acid sequence sharing at least about 95%, 96%, 97%, 98%, 99% or 100% sequence identity to the nucleic acid sequence set forth in SEQ ID NO:1. Another aspect of the invention provides an isolated nucleic acid comprising a nucleic acid sequence sharing at least about 95%, 96%, 97%, 98%, 99% or 100% sequence identity to the nucleic acid sequence set forth in SEQ ID NO:2.

A further aspect of the invention provides an isolated polypeptide comprising a nucleic acid sequence sharing at least about 95%, 96%, 97%, 98%, 99% or 100% sequence identity to the amino acid sequence set forth in SEQ ID NO:13. Another aspect of the invention provides an isolated polypeptide comprising a nucleic acid sequence sharing at least about 95%, 96%, 97%, 98%, 99% or 100% sequence identity to the nucleic acid sequence set forth in SEQ ID NO:14.

The invention further provides expression vectors comprising the polynucleotides operably linked to an expression control sequence, host cells (e.g., mammalian cells, bacterial cells such as E. coli, or yeast cells) comprising the expression vectors, and isolated polypeptides comprising an amino acid sequence encoded by the polynucleotides.

Another embodiment of the invention provides an isolated nucleic acid comprising the sequence set forth in SEQ ID NO:3, 4, 5, 6, 7, 8, 9, 10, 11, or 12.

These and other embodiments of the invention are set forth in greater detail in the detailed description that follows.

### BRIEF DESCRIPTION OF THE SEQUENCES

SEQ ID NO:1 sets forth a cDNA sequence encoding pig T1R1.

SEQ ID NO:2 sets forth a cDNA sequence encoding pig T1R3.

SEQ ID NO:3 sets forth a forward gene specific primer sequence that finds use for amplifying T1R3.

SEQ ID NO:4 sets forth a reverse gene specific primer sequence that finds use for amplifying T1R3.

SEQ ID NO:5 sets forth a forward degenerate primer sequence that finds use for amplifying T1R1.

SEQ ID NO:6 sets forth a reverse degenerate primer sequence that finds use for amplifying T1R1.

SEQ ID NO:7 sets forth a forward primer sequence that finds use for amplifying T1R3.

SEQ ID NO:8 sets forth a reverse primer sequence that finds use for amplifying T1R3.

SEQ ID NO:9 sets forth a reverse nested primer sequence that finds use for amplifying T1R3.

SEQ ID NO:10 sets forth a forward primer sequence that finds use for amplifying T1R1.

SEQ ID NO:11 sets forth a forward nested primer sequence that finds use for amplifying T1R1.

SEQ ID NO:12 sets forth a reverse primer sequence that finds use for amplifying T1R1.

SEQ ID NO:13 sets forth an amino acid sequence of pT1R1.

SEQ ID NO:14 sets forth an amino acid sequence of pT1R3.

### BRIEF DESCRIPTION OF THE DRAWINGS

FIG. 1 illustrates an alignment of T1R1 amino acid homologues from porcine (SEQ ID NO:15), feline (SEQ ID NO:16), human (SEQ ID NO:17), mouse (SEQ ID NO:18) and rat (SEQ ID NO:19). A consensus sequence based on this alignment is provided in the bottom row. The dark and medium gray highlighting represent identity and similarity, respectively.

FIG. 2 illustrates an alignment of T1R3 amino acid homologues from porcine (SEQ ID NO:20), feline (SEQ ID NO:21), human (SEQ ID NO:22), mouse (SEQ ID NO:23) and rat (SEQ ID NO:24). A consensus sequence based on this alignment is provided in the bottom row. The dark and medium gray highlighting represent identity and similarity, respectively.

FIG. 3 illustrates the amino acid sequence of pig T1R1 (SEQ ID NO:25). The predicted ligand binding domain is shaded and the predicted transmembrane (TM) regions are underlined.

FIG. 4 illustrates a ClustalW alignment of human (SEQ ID NO:26), mouse (SEQ ID NO:27) and pig (SEQ ID NO:28) T1R3 amino acid sequences. Underlined regions indicate membrane spanning domains. Membrane spanning regions for human and pig T1R3 were based upon UniProtKB/Swiss=Prot entries Q7RTX0 and Q91VA4, respectively. Transmembrane regions for pig T1R3 were determined using TMHMM. The yellow highlighted sequence denotes a region where a membrane spanning domain for pig T1R3 was not predicted. * indicates conserved amino acid residues.

FIG. 5 illustrates the amino acid sequence of pig T1R3 (SEQ ID NO:29). The predicted ligand binding domain is shaded and the predicted transmembrane (TM) regions are underlined.

### DETAILED DESCRIPTION OF THE INVENTION

### I. Introduction

The present invention is based on the identification of nucleic acids encoding the pig taste receptors pT1R1 and pT1R3 and polypeptides encoded by the pT1R1 and pT1R3 nucleic acids.

The nucleic acids and polypeptides described herein can be used to characterize cellular processes for umami taste response in pigs. The nucleic acids and polypeptides can also be used in methods to identify compounds that modulate taste signal transduction, i.e., to screen for modulators (e.g., activators, inhibitors, stimulators, enhancers, agonists, and antagonists) of these novel umami taste receptors comprising pT1R1, pT1R3, or a combination thereof. Such modulators of umami taste transduction are useful for pharmacological and genetic modulation of umami taste signaling pathways, and for the discovery of novel umami taste ligands. These methods of screening can be used to identify high affinity agonists and antagonists of umami taste cell activity. These modulatory compounds can then be used in the animal feed and veterinary pharmaceutical industries to customize flavor, for example, for porcine foodstuffs, medicines and nutritional supplements.

Thus, the invention provides assays for taste modulation, where the pT1R1- or pT1R3-comprising receptor acts as a direct or indirect reporter molecule for the effect of modulators on umami taste transduction. G protein coupled receptors (GPCRs) can be used in assays, e.g., to measure changes in ligand binding, G protein binding, regulatory molecule binding, ion concentration, membrane potential, current flow, ion flux, transcription, signal transduction, receptor-ligand interactions, neurotransmitter and hormone release; and second messenger concentrations, in vitro, in vivo, and ex vivo. In one embodiment, a receptor comprising pT1R1, pT1R3, or a combination thereof can be used as an indirect reporter via attachment to a second reporter molecule such as green fluorescent protein (see, e.g., Mistili & Spector, Nature Biotechnology 15: 961-964 (1997)). In another embodiment, a receptor comprising T1R3 is recombinantly expressed in cells, and modulation of taste transduction via GPCR activity is assayed by measuring changes in Ca²⁺ levels.

Methods of assaying for modulators of taste transduction include in vitro ligand binding assays using receptors comprising pT1R1, pT1R3 or a combination thereof, portions thereof such as the extracellular domain, or chimeric proteins comprising one or more domains of T1R3, and in in vivo (cell-based and animal) assays such as oocyte T1R3 receptor expression; tissue culture cell T1R3 receptor expression; transcriptional activation of T1R3; phosphorylation and dephosphorylation of GPCRs; G protein binding to GPCRs; ligand binding assays; voltage, membrane potential and conductance changes; ion flux assays; changes in intracellular second messengers such as cAMP and inositol triphosphate; changes in intracellular calcium levels; and neurotransmitter release.

Such compounds can be used to increase the palatability of feed, supplements, or medicaments (e.g., including oral vaccines and other medications). For example the compounds can be used to increase the palatability of complete feeds; milk replacers; mineral or/and vitamin mixes, protein concentrates, mineral feed, electrolyte supplements, amino acid supplements, feed additives such as, e.g., single or mixes of organic and inorganic acids, sylage inoculants, mold inhibitors, antioxidants, direct fed microbials, yeasts, pre and pro biotics, enzymes, pigments, colorants, antibiotics, growth promoters, toxin binders, clays, aluminosilicates, anti-caking agents, pellet binders, minerals, vitamins, single amino acids (including D- and L-amino acids), nucleotides, inulin, oligosaccharides and their derivatives (e.g., fructooligosaccharides, mananooligosaccharides and the like), glycerin, flavours and other palatability enhancers; feed ingredients such as, e.g., cereals, cereal derivatives, heat treated cereals, cereal byproducts such as cereal distillers' dried grains with solubles (DDGS) and mixtures thereof; such as, for example corn and corn derivatives, corn byproducts, oats and oat derivatives, barley, barley byproducts, wheat, wheat byproducts, rice, rice byproducts, sorghum, rye, extruded corn, extruded wheat, extruded barley, and the like, legumes and other plant proteins such as soya and all soya derivatives, lupin, sunflower, rapeseed, canola, cottonseed, peanut, peas, horsebeans, lentils, linseed, palmkernel, vetch, bitter vetch and potato meals and protein concentrates, wheat gluten, whole acorn, tapioca, molasses, sweet potato, and mixtures thereof; animal proteins such as, e.g., animal meals including fish meal, meat meal, blood meal, animal plasma, hemoglobin, poultry slaughtering byproduct, feather meal, and mixtures thereof; milk derivatives such as casein, skimmed milk, sweet and acid whey powders, glycerin, fiber sources such as, e.g., wheat bran, alfalfa, alfalfa derivatives such as hay, rice bran, wheat shorts and wheat middlings, barley sprouts, hulls (from cotton, oats, sunflower, soybean), carob bean, olive pulp, grape seed, grape pulp, cereal straw, citrus pulp and sugar beet pulp, and mixtures thereof; fats and oils and all their mixtures: animal fats (lard, butter, tallow, schmaltz or chicken fat, fish oils such as, e.g., oils from cod, menhadden, and the like) and vegetable fats (e.g., oils extracted from soybean, rape, olive, palm and coconut, oleins from soybean, and sunflower, and soybean lecithins); liquid diets (i.e., diets comprising a mixture of complete feed and/or feed supplements with water); drinking water or drinking water supplements such as, for example, electrolytes, sugars, amino acids, nucleotides and/or other nutrients, feed ingredients, concentrates, other supplements or additives.

The nucleic acids and polypeptides described herein can also be used to identify pigs with a higher or lower threshold of umami taste response. Identification of such pigs can assist farmers in selecting and developing breeding stocks of pigs. The nucleic acids described herein can also be used to produce transgenic pigs.

### II. Definitions

A “T1R family taste receptor” or an “umami T1R taste receptor” interchangeably refer to a member of the T1R family of G protein coupled receptors expressed in taste cells and involved in conveying savory or umami taste signaling or sensations. Structurally, umami T1R family taste receptors include heterodimeric, homodimeric, and monomeric receptors comprising T1R1 and T1R3 subunits. Functionally, an umami T1R family taste receptor binds to one or more savory or umami ligands, including, e.g., naturally occurring and/or artificial umami tasting molecules, e.g., monosodium glutamate (MSG), guanine monophosphate (GMP), inositol monophosphate (IMP), ribonucleotides, or combinations thereof, which are agonists that elicit increased intracellular secondary messengers, e.g., intracellular calcium and inositol triphosphate (IP₃) release. See, e.g., Medler, Crit. Rev Eukaryot Gene Expr (2008) 18(2):125-137; Roper, Pflugers Arch (2007) 454(5):759-76; Palmer, Mol Intern (2007) 7(2):87-98; and Reed, et al., Physiol Behav (2006) 88(3):215-26. “pT1R,” or “pT1R1,” or “pT1R3” as used herein refer to polypeptide components of the porcine taste receptors. “pTAS1R,” or “pTAS1R1,” or “pTAS1R3” as used herein refer to nucleic acids encoding porcine taste receptors.

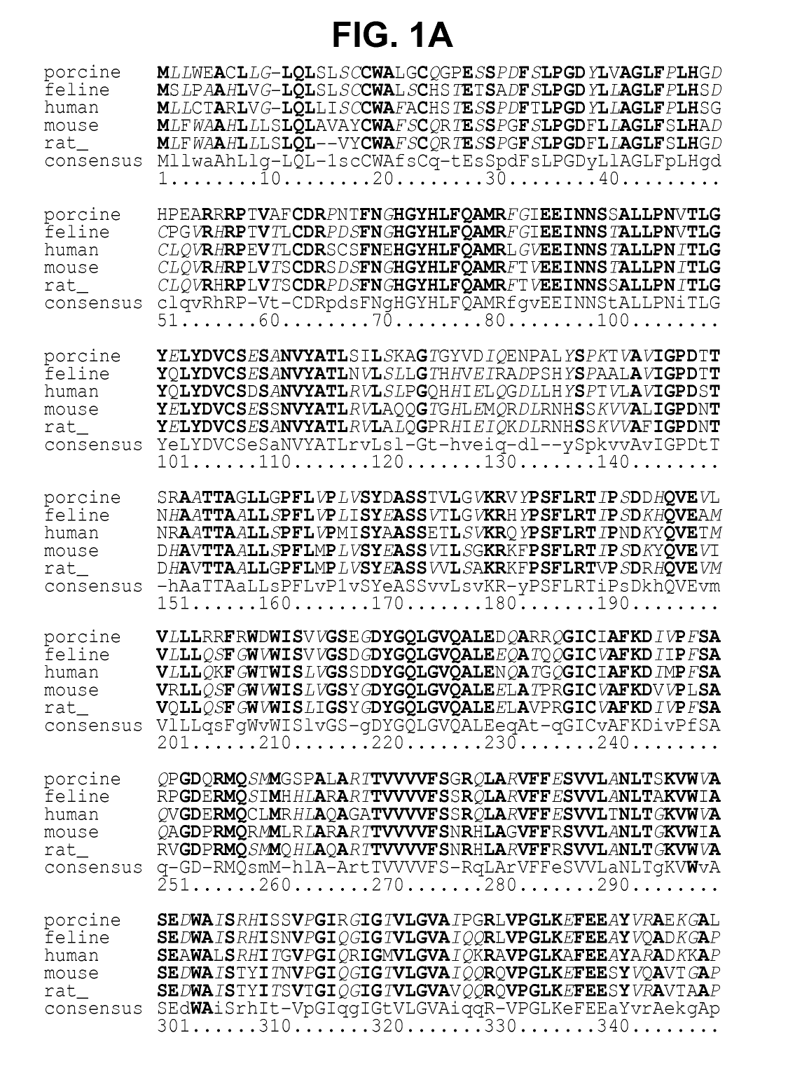

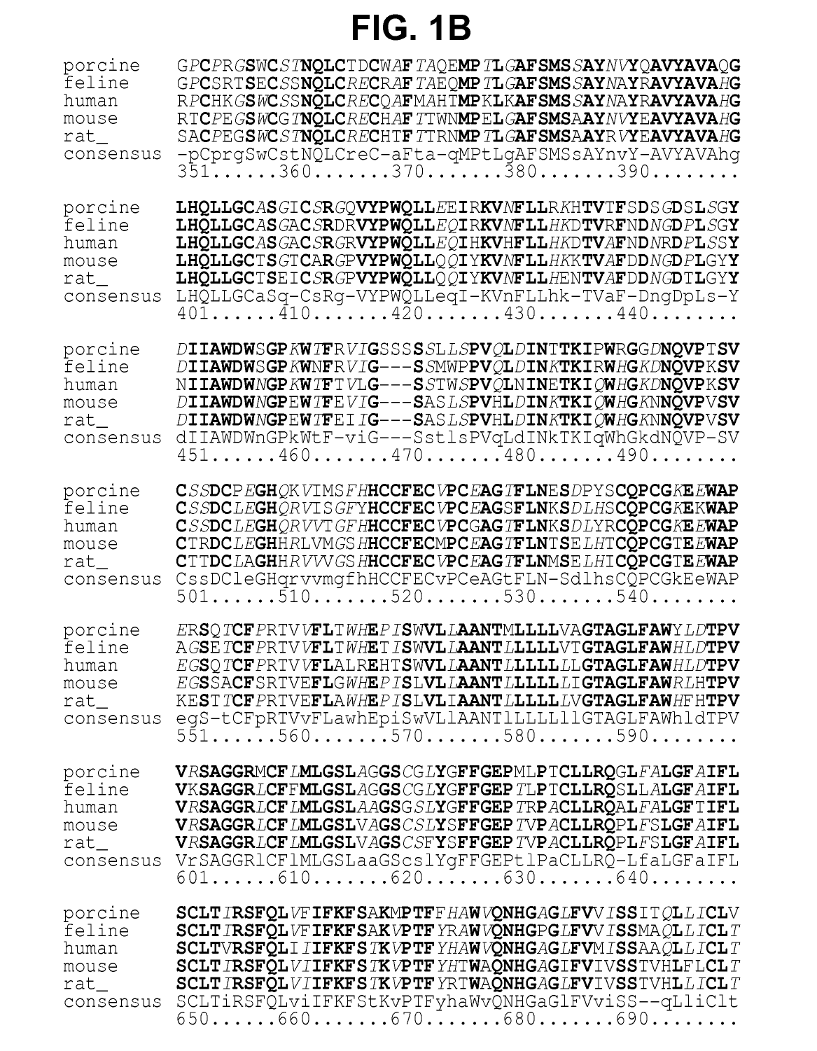

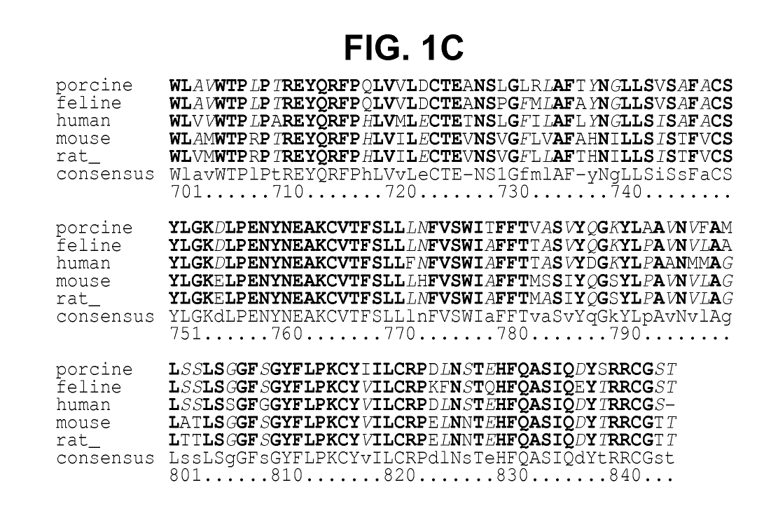

A porcine T1R1 nucleic acid sequence (“pTAS1R1”) shares at least 90%, 95%, 96%, 97%, 98%, 99% or 100% nucleic acid sequence identity with a nucleic acid sequence of SEQ ID NO:1. A porcine T1R1 amino acid sequence shares at least 90%, 95%, 96%, 97%, 98%, 99% or 100% amino acid sequence identity with an amino acid sequence encoded by a nucleic acid sequence of SEQ ID NO:1 or an amino acid sequence of SEQ ID NO:13. Sequence identity can be measured using the default parameters or settings of a known algorithm (e.g., BLAST, ALIGN) over a sequence length of at least about 50, 100, 150, 200, 250, 300, 500 or 800 nucleic acid bases or amino acid residues, or over the full length of the sequence. A porcine T1R1 amino acid sequence has a predicted secondary structure that possesses seven transmembrane helices that form a transmembrane domain having 86 and 87% sequence identity with the corresponding transmembrane regions of T1R1 from human and mouse, respectively. The extracellular domain of pT1R1 has 79 and 81% sequence identity with the corresponding extracellular domains of T1R1 from human and mouse, respectively. A porcine T1R1 receptor nucleic acid sequence shares about 85% sequence identity with the T1R1 receptor from Bos taurus (GenBank Accession No. XM_{—}601773.2) and Felis catus (BWB26448); about 84% sequence identity with the T1R1 receptor from Canis familiaris (XM_{—}546753.2) and Equus caballus (XM_{—}001496112.1); about 81% sequence identity with the T1R1 receptor from Homo sapiens (BK000153.1), Pan troglodyte (XM_{—}525169.2), and Macaca mulatta (XM_{—}001116977.1); and about 79% sequence identity with the T1R1 receptor from rattus (NM_{—}053305.1) and murine (NM_{—}031867). A porcine T1R1 receptor amino acid sequence shares about 80% sequence identity with the T1R1 receptor from Bos taurus (GenBank Accession No. XP_{—}601773.2) and Felis catus (BWB26448); about 75% sequence identity with the T1R1 receptor from Homo sapiens (AAL91359); and about 71% sequence identity with the T1R1 receptor from rattus (AAD18069) and murine (AAK51603). Based on the alignment provided in FIG. 1, those of skill will appreciate that positions with residues conserved throughout different species (e.g., residues represented by a capital letter in the consensus sequence) should not be altered. However, positions with non-conserved residues (i.e., no highlighting) can tolerate amino acid substitution or deletion without altering the function of the taste receptor. Positions with conservatively substituted or similar residues (i.e., light grey highlighting) can tolerate conservative amino acid substitutions without altering the function of the taste receptor.

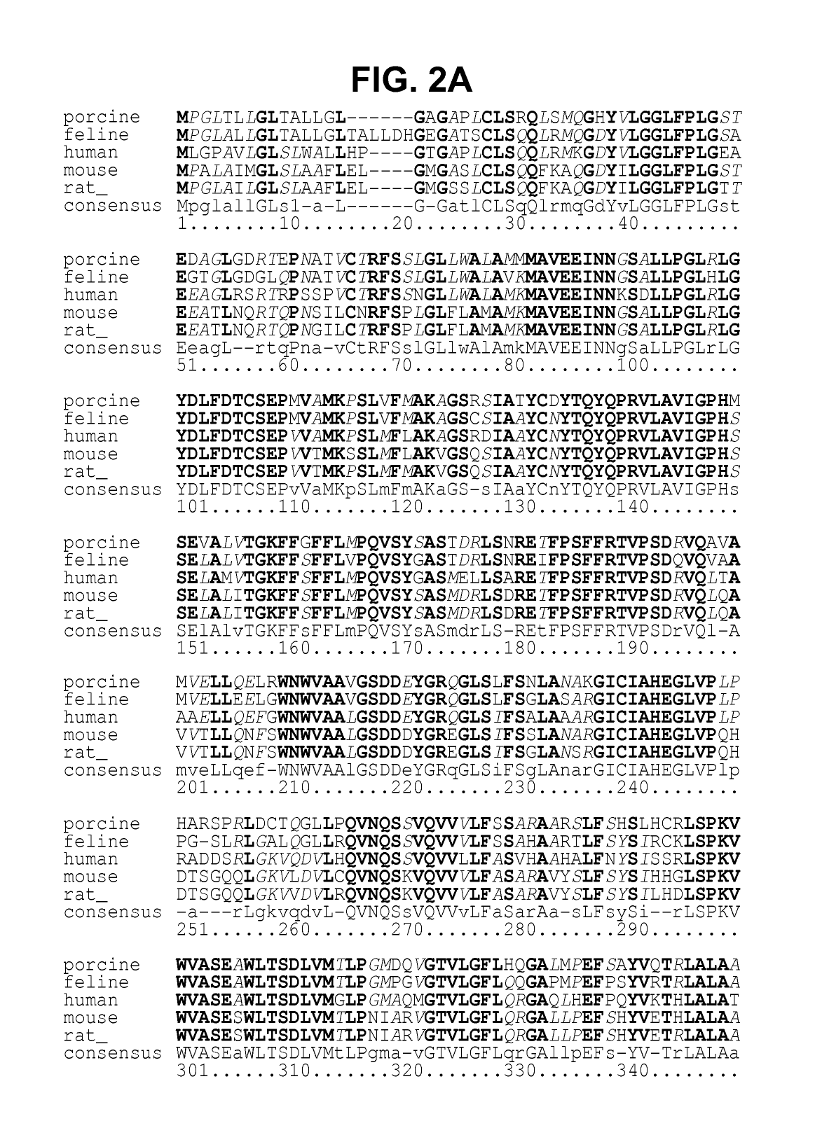

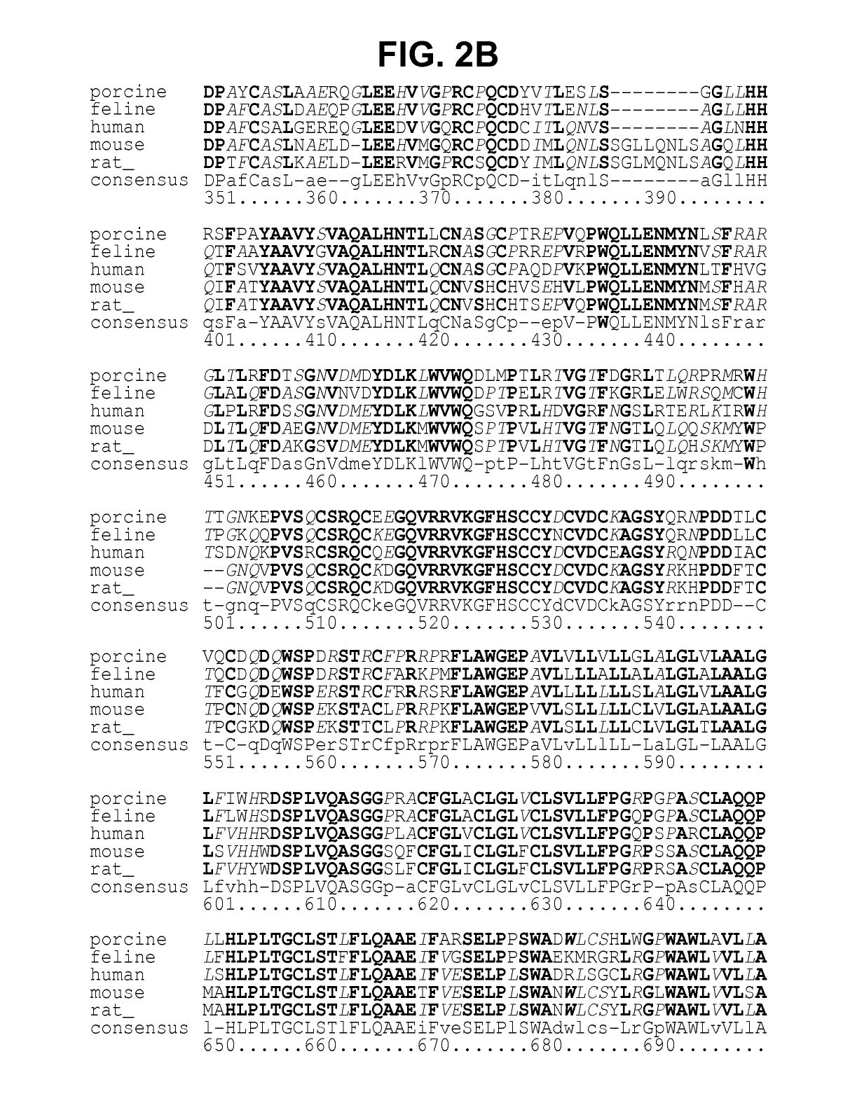

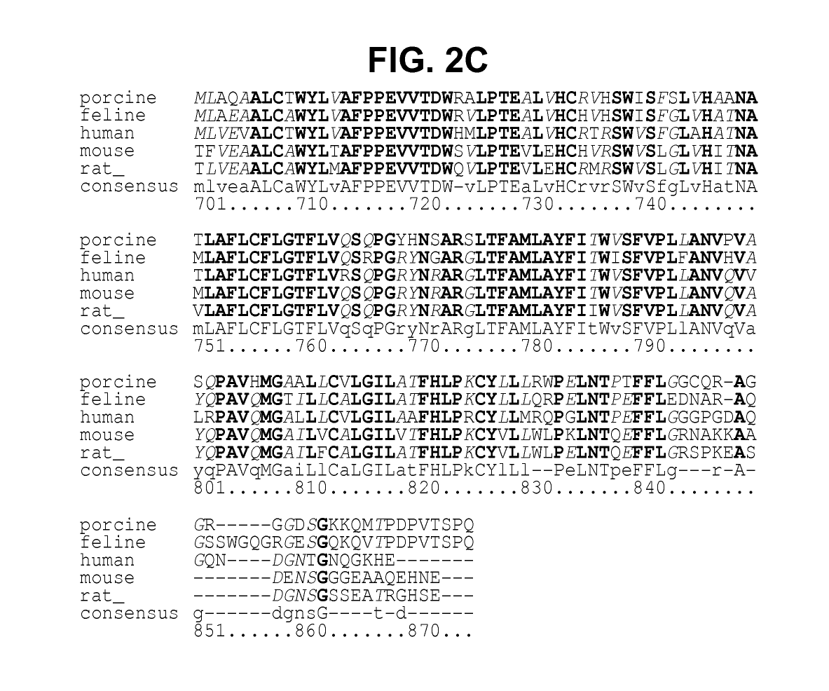

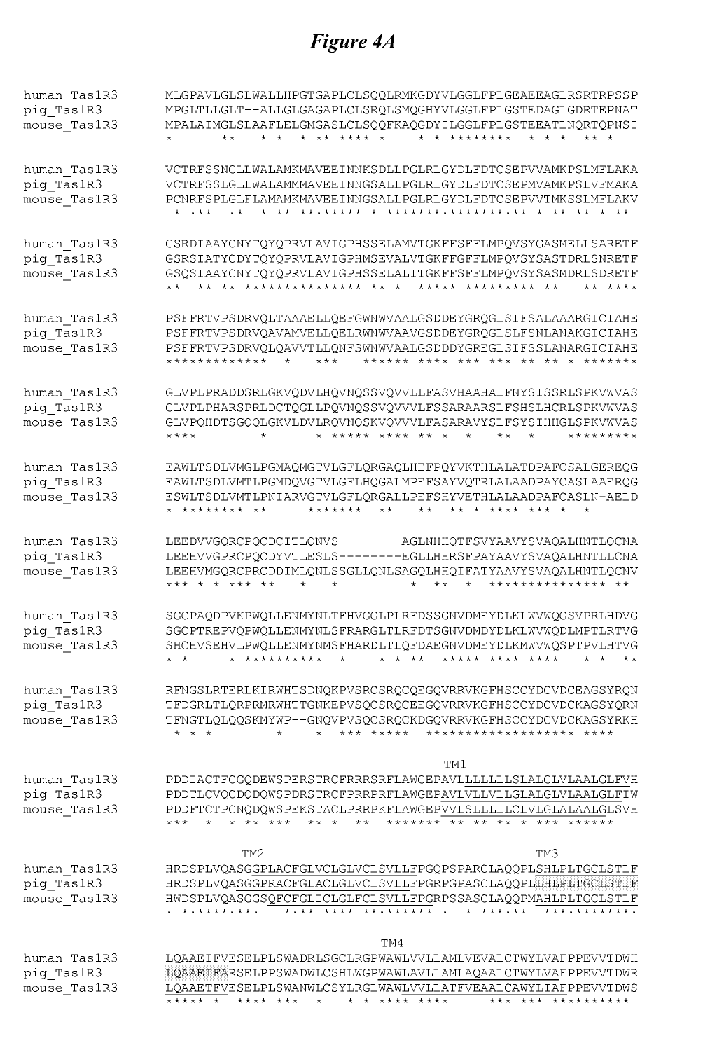

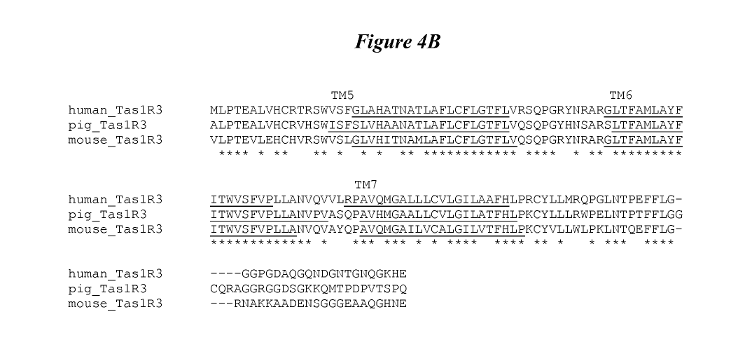

Structurally, a porcine T1R3 nucleic acid sequence (“pTAS1R3”) shares at least 90%, 95%, 97%, 98%, 99% or 100% nucleic acid sequence identity with a nucleic acid sequence of SEQ ID NO:2. A porcine T1R3 amino acid sequence shares at least 90%, 95%, 96%, 96%, 97%, 98%, 99% or 100% amino acid sequence identity with an amino acid sequence encoded by a nucleic acid sequence of SEQ ID NO:2 or an amino acid sequence of SEQ ID NO:14. Sequence identity can be measured using the default parameters or settings of a known algorithm (e.g., BLAST, ALIGN) over a sequence length of at least about 50, 100, 150, 200, 250, 300, 500 or 800 nucleic acid bases or amino acid residues, or over the full length of the sequence. The amino acid coding sequences of pT1R3 has a predicted secondary structure that possesses seven transmembrane helices that form a transmembrane domain possessing 82% and 77% homology with the corresponding transmembrane domains of the T1R3 from human and mouse, respectively. The amino acid sequence of T1R3 has a large extracellular domain that has 75% and 74% sequence identity with the extracellular domain of the T1R3 from human and mouse, respectively. A porcine T1R3 receptor nucleic acid of SEQ ID NO:2 shares about 99% sequence identity with the T1R3 receptor from Sus scrofa (GenBank Accession Nos. AB162129; AB162128; and NM_{—}01113288); about 84% sequence identity with the T1R3 receptor from Bos taurus (XM_{—}588865) and Felis catus (NM_{—}01114547); about 82% sequence identity with the T1R3 receptor from Homo sapiens (NM_{—}152228), and about 74% sequence identity with the T1R3 receptor from rattus (AF46324) and murine (NM_{—}031872). A porcine T1R3 receptor amino acid sequence shares about 77% sequence identity with the T1R3 receptor from Bos taurus (XP_{—}588865) and Felis catus (NP_{—}01108019); about 74% sequence identity with the T1R3 receptor from Homo sapiens (NP_{—}689414); and about 72% sequence identity with the T1R3 receptor from rattus (AAM10636) and murine (AAK5537). Based on the alignment provided in FIGS. 2 and 4, those of skill will appreciate that positions with residues conserved throughout different species (e.g., residues represented by a capital letter in the consensus sequence) should not be altered. However, positions with non-conserved residues (i.e., no highlighting) can tolerate amino acid substitution or deletion without altering the function of the taste receptor. Positions with conservatively substituted or similar residues (i.e., light grey highlighting) can tolerate conservative amino acid substitutions without altering the function of the taste receptor.

With respect to truncated T1R1 or T1R3 receptor sequences or fragments, ligand binding assays that do not require evaluation of intracellular signaling can be performed using truncated sequence segments comprising the ligand binding domain. See, Table 3. The signal peptide, intracellular cytoplasmic domain, and the region from TM1 to TM7 can be optionally excluded from the truncated receptor fragments when conducting ligand binding assays that do not require intracellular signaling. When performing functional assays that evaluate the ability of a ligand to induce signaling through a T1R1 or T1R3 receptor, the signal peptide can optionally be excluded. In some embodiments, portions of the ligand binding domain or intracellular C-terminal domain can be excluded, if ligand binding or intracellular signaling functions are not substantially affected.

“Umami” as used herein refers to the savory taste response or the amino acid taste response (see, e.g., a taste sensation responsive to savory ingestible products). The taste sensation is described based on the reactivity of various ligands with the “umami receptor,” i.e., a receptor comprising pT1R1, pT1R3, or combinations thereof. Examples of ligands capable of eliciting an “umami” sensation include, including without limitation, monosodium glutamate (MSG), GMP, IMP, or combinations thereof.

“Porcine” or “pig” as used herein refers to a domesticated (purebred or crossbreeds) or wild mammal of the genus sus, (including for example, sus barbatus, sus bucculentus, sus cebifrons, sus celebensis, sus domestica, sus falconeri, sus heureni, sus hysudricus, sus philippensis, sus salvanius, sus scrofa, sus strozzi, sus timoriensis, sus verrucosus). Specific breeds of pigs include, e.g., American Landrace, American Yorkshire, Angeln Saddleback, Arapawa Island, Ba Xuyen, Bantu, Bazna, Beijing Black, Belarus Black Pied, Belgian Landrace, Bentheim Black Pied, Berkshire, Black Slavonian, British Landrace, British Lop, Bulgarian White, Cantonese, Chester White, Czech Improved White, Danish Landrace, Dermantsi Pied, Duroc, Dutch Landrace, Fengjing, Finnish Landrace, French Landrace, German Landrace, Gloucestershire Old Spots, Guinea Hog, Hampshire, Hereford, Hezuo, Iberian, Italian Landrace, Jinhua, Kele, Krskopolje, Kunekune, Lacombe, Large Black, Large Black-white, Large White, Lithuanian Native, Mangalitsa, Meishan, Middle White, Minzhu, Mong Cai, Mukota, Mora Romagnola, Moura, Mulefoot, Neijiang, Ningxiang, Norwegian Landrace, Ossabaw Island, Oxford Sandy and Black, Philippine Native, Pietrain, Poland China, Red Wattle, Saddleback, Spotted, Swabian-Hall, Swedish Landrace, Tamworth, Thuoc Nhieu, Tibetan, Turopolje, Vietnamese Potbelly, Welsh, Wuzhishan, and Yorkshire.

T1R proteins have “G protein coupled receptor activity,” e.g., they bind to G proteins in response to extracellular stimuli, such as ligand binding (e.g., sweet ligands or umami ligands), and promote production of second messengers such as IP₃, cAMP, and Ca²⁺ via stimulation of enzymes such as phospholipase C and adenylate cyclase. Such activity can be measured in a heterologous cell, by coupling a G protein coupled receptor, or GPCR, (or a chimeric GPCR) to either a G protein or promiscuous G protein such as Gα₁₅ or Gα16 Gαz and an enzyme such as PLC, and measuring increases in intracellular calcium (Offermans & Simon, J. Biol. Chem. 270:15175-15180 (1995)).

T1R proteins, when activated by ligand binding, elicit an “elevation of cAMP,” or “increase in cAMP” levels relative to a control, e.g., 10%, 20%, 30%, 40%, 50%, 60%, 70%, 80%, 90%, 100%, or higher relative to a control. Receptor activity or T1R binding of ligand can also be detected based on “elevated Ca²⁺ levels,” or “increase in Ca²⁺ levels,” or “changes in Ca²⁺ flux” relative to a control, e.g., 10%, 20%, 30%, 40%, 50%, 60%, 70%, 80%, 90%, 100%, or higher relative to a control. Ca²⁺ flux can be measured, e.g., by recording ligand-induced changes in [Ca²⁺]i using fluorescent Ca²⁺-indicator dyes and fluorometric imaging.

GPCRs have transmembrane, extracellular and cytoplasmic domains that can be structurally identified using methods known to those of skill in the art, such as sequence analysis programs that identify hydrophobic and hydrophilic domains (see, e.g., Kyte & Doolittle, J. Mol. Biol. 157:105-132 (1982)). Such domains are useful for making chimeric proteins and for in vitro assays of the invention (see, e.g., WO 94/05695 and U.S. Pat. No. 5,508,384).

The phrase “functional effects” in the context of assays for testing compounds that modulate activity (e.g., signal transduction) of taste receptor or protein of the invention includes the determination of a parameter that is indirectly or directly under the influence of a GPCR or a taste receptor, e.g., a physical, phenotypic, or chemical effect, such as the ability to transduce a cellular signal in response to external stimuli such as ligand binding, or the ability to bind a ligand including binding activity and signal transduction. “Functional effects” include in vitro, in vivo, and ex vivo activities.

“Determining the functional effect” as used herein means assaying for a compound that increases or decreases a parameter that is indirectly or directly under the influence of a pT1R GPCR protein or a taste receptor, e.g., a umami taste receptor comprising one or more pT1R GPCR proteins, e.g., physical and chemical or phenotypic effect. Functional effects include, e.g., changes in Ca²⁺ flux, increase in cAMP levels, increase in IP₃ levels and protein kinase activation.

“Inhibitors,” “activators,” and “modulators” of T1R family polynucleotide and polypeptide sequences and T1R family taste receptors are used herein to refer to activating, inhibitory, or modulating molecules identified using in vitro and in vivo assays of T1R polynucleotide and polypeptide sequences and T1R family taste receptors, including monomeric, homodimeric and heterodimeric receptors. Samples or assays comprising the T1R family of taste receptors treated with a potential activator, inhibitor, or modulator are compared to control samples without the inhibitor, activator, or modulator to examine the extent of inhibition, activation or modulation. Control samples (untreated with inhibitors, activators, or modulators) are assigned a relative protein activity value of 100%.

“Inhibitors” are compounds that, e.g., bind to, partially or totally block activity, decrease, prevent, delay activation, inactivate, desensitize, or down-regulate the activity or expression of the T1R family of taste receptors, e.g., antagonists Inhibition of a T1R family receptor is achieved when the activity value relative to the control (untreated with inhibitors) is about 95%, 90%, 85%, 80%, 75%, 70%, 65%, 60%, 55%, 50%, 45%, 40%, 35%, 30%, 25%, 20%, 15%, 10%, 5% or 0%. In some embodiments, inhibition of a T1R family receptor is achieved when the activity value relative to the control (untreated with inhibitors) is 600-1000% lower or 1000-3000% lower.

“Activators” are compounds that increase, open, activate, facilitate, enhance activation, sensitize, agonize, or up-regulate a T1R family taste receptor, e.g., agonists. Activation of a T1R family receptor is achieved when the activity value relative to the control (untreated with activators) is 110%, 120%, 130%, 140%, 150%, 160%, 170%, 180%, 190% m 200%, 225%, 250%, 275%, 300%, 325%, 350%, 375%, 400%, 425%, 450%, 475% or 500% or more (i.e., 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5 fold or more) higher relative to the control. In some embodiments, activation of a T1R family receptor is achieved when the activity value relative to the control (untreated with activators) is 600-1000% higher or 1000-3000% higher.

Inhibitors, activators, or modulators also include genetically modified versions of T1R family taste receptors, e.g., versions with altered activity, as well as naturally occurring and synthetic ligands, antagonists, agonists, antibodies, antisense molecules, ribozymes, small chemical molecules and the like. Such assays for inhibitors and activators include, e.g., expressing T1R family taste receptors in vitro, in cells, or cell membranes, applying putative modulator compounds, and then determining the functional effects on activity, as described above. Ligands for the taste receptors, e.g., sweet taste receptor ligands or umami receptor ligands can modulate taste signal transduction by acting as extracellular ligands for the G protein coupled receptor and activating the receptor. In other embodiments, compounds that modulate taste signal transduction are molecules that act as intracellular ligands of the receptor, or inhibit or activate binding of an extracellular ligand, or inhibit or activate binding of intracellular ligands of the receptor.

The term “test compound” or “drug candidate” as used herein describes any molecule either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulate taste. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons. Typically a small organic molecule has a molecular weight of less than about 2000 daltons, less than 1500 daltons, less than 1000 daltons, less than 900 daltons, less than 800 daltons, less than 700 daltons, less than 600 daltons, less than 500 daltons, less than 400 daltons, less than 300 daltons, less than 200 daltons or less than 100 daltons.

“Biological sample” include sections of tissues such as biopsy and autopsy samples, and frozen sections taken for histologic purposes. Such samples include blood, sputum, tissue (e.g., from the tongue), cultured cells, e.g., primary cultures, explants, and transformed cells, stool, urine, etc. A biological sample is typically obtained from a porcine.

A “heterodimeric T1R receptor” as used herein refers to a dimeric receptor comprising two different polypeptide subunits, e.g., two different polypeptides such as pT1R1 and pT1R3. The polypeptide subunits may be associated via either covalent bond or a noncovalent bond. Suitable bonds includes linkers or a chemical bond, or non-covalent, e.g., ionic, van der Waals, electrostatic, or hydrogen bonds linkages. The heterodimeric receptors of the invention function when co-expressed in the same cell, preferably when co-expressed so that they form a heterodimer, either covalently or non-covalently linked. For example, T1R1 and T1R3 form a heteromeric receptor.

A “homodimeric T1R receptor” as used herein refers to a dimeric receptor comprising two of the same polypeptide subunits, e.g., two pT1R1 polypeptides or two pT1R3 polypeptides, where the molecules are associated via either covalent, e.g., through a linker or a chemical bond, or non-covalent, e.g., ionic, van der Waals, electrostatic, or hydrogen bond linkages. The homodimeric T1R receptors of the invention (e.g., homodimeric T1R1 or T1R3) function when co-expressed in the same cell, preferably when co-expressed so that they form a homodimer, either covalently or non-covalently linked.

A “monomer” is a receptor comprising one polypeptide subunit, e.g., one pT1R1 polypeptide or one pT1R3 polypeptide.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 80% identity, for example, about 85%, 90%, 91%, 92%, 93%, 94%, 95%, 96%, 97%, 98%, 99%, or higher identity over a specified region to a reference sequence (e.g., nucleotide sequences SEQ ID NO:1 or 2 or amino acid sequences SEQ ID NO:13 or 14), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Identity exists over a region that is 100, 200, 300, 400, 500, 600, 700 or 800 amino acids or nucleotides in length, or over the full length of the sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, or 30 to 500 or 40 to 400, usually about 50 to about 200, or 60 to 180 or 70 to 170 or 80 to 160 or 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1994-2008)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (on the Worldwide Web at ncbi.nlm.nih.gov). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, enantiomers (D- and L-forms), and achiral amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetic” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3.sup.rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., extracellular domains, transmembrane domains, and cytoplasmic domains. Domains are portions of a polypeptide that form a compact unit of the polypeptide and are typically 15 to 350 amino acids long. Typical domains are made up of sections of lesser organization such as stretches of β-sheets and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

“Nucleic acid” refers to deoxyribonucleotides or ribonucleotides and polymers thereof in either single- or double-stranded form, as well as the complements of any such sequence. Also included are DNA, cDNA, RNA, polynucleotides, nucleotides, and the like. The term encompasses nucleic acids containing known nucleotide analogs or modified backbone residues or linkages, which are synthetic, naturally occurring, and non-naturally occurring, having similar binding properties as the reference nucleic acid, and that are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein, e.g., a porcine taste receptor, comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include 30-40 cycles of the following conditions: a denaturation phase of 90° C.-95° C. for 30 sec-2 min., an annealing phase lasting 30 sec-2 min, and an extension phase of about 72° C. for 1-2 min. Protocols and guidelines for low and high stringency amplification reactions are provided, e.g., in Innis et al., PCR Protocols, A Guide to Methods and Applications (1990).

### III. Nucleic Acids Encoding pT1R

One embodiment of the invention provides nucleic acids encoding pT1R1 and pT1R3, including monomers, homdimers, and heterodimers thereof.

A. General Recombinant DNA Methods

This invention relies on routine techniques in the field of recombinant genetics. Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual, (3rd ed., 2001, Cold Spring Harbor Laboratory Press); Kriegler, Gene Transfer and Expression: A Laboratory Manual (1990); and Current Protocols in Molecular Biology (Ausubel et al., eds., 1994-2008, Wiley Interscience).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

B. Cloning Methods for the Isolation of Nucleotide Sequences Encoding pT1R

In general, the nucleic acid sequences encoding pT1R and related nucleic acid sequence homologues are cloned from cDNA and genomic DNA libraries or isolated using amplification techniques with oligonucleotide primers. For example, pT1R sequences are typically isolated from nucleic acid (genomic or cDNA) libraries by hybridizing with a nucleic acid probe, the sequence of which can be derived from SEQ ID NO: 1, 2, 3, or a complement or a subsequence thereof pT1R RNA and cDNA can be isolated from any porcine.

pT1R polymorphic variants and alleles that are substantially identical to pT1R can be isolated using pT1R nucleic acid probes and oligonucleotides under stringent hybridization conditions, by screening libraries. Alternatively, expression libraries can be used to clone T1R polymorphic variants and alleles, by detecting expressed variants and alleles immunologically with antisera or purified antibodies made against the core domain of pT1R which also recognize and selectively bind to the pT1R variants and alleles.

To make a cDNA library, pT1R mRNA may be purified from any porcine. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 1-8 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., PNAS USA., 72:3961-3965 (1975).

An alternative method of isolating pT1R nucleic acids and their homologues combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of pT1R directly from mRNA, from cDNA, from genomic libraries or cDNA libraries. Degenerate oligonucleotides can be designed to amplify pT1R homologues using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of pT1R encoding mRNA in physiological samples, for nucleic acid sequencing, or for other purposes. Genes amplified by the PCR reaction can be purified from agarose gels and cloned into an appropriate vector.

Amplification techniques using primers can also be used to amplify and isolate pT1R DNA or RNA. For example, nucleic acids encoding pT1R or fragments thereof may be obtained by amplification of a porcine cDNA library or reverse transcribed from porcine RNA using primer pairs. In some embodiments, an isolated pT1R1 nucleic acid is amplified from porcine genomic DNA, mRNA or cDNA template using a forward primer selected from the group consisting of SEQ ID NO:5, SEQ ID NO:10 and SEQ ID NO:11, and a reverse primer selected from the group consisting of SEQ ID NO:6 and SEQ ID NO:12. In some embodiments, an isolated pT1R3 nucleic acid is amplified from porcine genomic DNA, mRNA or cDNA template using a forward primer selected from the group consisting of SEQ ID NO:3, SEQ ID NO:7, and a reverse primer selected from the group consisting of SEQ ID NO:4, SEQ ID NO:8 and SEQ ID NO:9.

These primers can be used, e.g., to amplify either the full length sequence or a probe of one to several hundred nucleotides, which is then used to screen a cDNA library for full-length pT1R.

Gene expression of pT1R can also be analyzed by techniques known in the art, e.g., reverse transcription and amplification of mRNA, isolation of total RNA or poly A+ RNA, northern blotting, dot blotting, in situ hybridization, RNase protection, probing DNA microchip arrays, and the like.

Synthetic oligonucleotides can be used to construct recombinant pT1R genes for use as probes or for expression of protein. This method is performed using a series of overlapping oligonucleotides usually 40-120 by in length, representing both the sense and non-sense strands of the gene. These DNA fragments are then annealed, ligated and cloned. Alternatively, amplification techniques can be used with precise primers to amplify a specific subsequence of the pT1R gene. The specific subsequence is then ligated into an expression vector. pT1R chimeras can be made, which combine, e.g., a portion of pT1R with a portion of a heterologous pT1R to create a chimeric, functional pT1R.

The gene for pT1R is typically cloned into intermediate vectors before transformation into prokaryotic or eukaryotic cells for replication and/or expression. These intermediate vectors are typically prokaryote vectors, e.g., plasmids, or shuttle vectors. Isolated nucleic acids encoding pT1R proteins comprise a nucleic acid sequence encoding a pT1R protein and subsequences, interspecies homologues, alleles and polymorphic variants thereof. In some embodiments, the isolated nucleic acid encoding a pT1R protein is SEQ ID NO: 1, 2 or a complement thereof.

C. Expression of pT1R

To obtain high level expression of a cloned gene, such as those cDNAs encoding pT1R, one typically subclones pT1R into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing the pT1R protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the pT1R encoding nucleic acid in host cells. A typical expression cassette thus contains a promoter operably linked to the nucleic acid sequence encoding pT1R and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST (SEQ ID NO:30) and LacZ (SEQ ID NO:31). Epitope tags can also be added to recombinant proteins to provide convenient methods of isolation, e.g., c-myc (SEQ ID NO:32).

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of pT1R protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing pT1R.

After the expression vector is introduced into the cells, the transfected cells are cultured under conditions favoring expression of pT1R, which is recovered from the culture using standard techniques identified below.

D. Purification of pT1R Protein

Either naturally occurring or recombinant pT1R can be purified for use in functional assays. Naturally occurring pT1R are purified, e.g., from porcines and any other source of a pT1R variant or allele. Recombinant pT1R is purified from any suitable expression system.

pT1R may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant pT1R is being purified. For example, proteins having established molecular adhesion properties can be reversibly fused to pT1R. With the appropriate ligand, pT1R can be selectively adsorbed to a purification column and then freed from the column in a relatively pure form. The fused protein is then removed by enzymatic activity. Finally, pT1R could be purified using immunoaffinity columns.

### IV. Detecting pT1R Nucleic Acid Sequences

In one embodiment of the invention, methods of detecting pT1R1 and pT1R3 sequences are provided, e.g., to identify porcines carrying the pT1R1 and pT1R3 sequences disclosed herein. Determination of the presence or absence of a particular pT1R1 or pT1R3 sequence is generally performed by analyzing a nucleic acid sample that is obtained from the porcine. Often, the nucleic acid sample comprises genomic DNA. It is also possible to analyze RNA samples for the presence or absence of pT1R1 and pT1R3 sequences.

In some embodiments, the pT1R1 and pT1R3 nucleic acids are detected using oligonucleotide primers and/or probes (i.e., primers and probes that amplify and detect SEQ ID NOS: 1, 2, or subsequences thereof) using methods known in the art. For example, a nucleic acid encoding pT1R1, or fragments thereof, may be amplified from porcine genomic DNA, mRNA or cDNA template using a forward primer selected from the group consisting of SEQ ID NO:5, SEQ ID NO:10 and SEQ ID NO:11, and a reverse primer selected from the group consisting of SEQ ID NO:6 and SEQ ID NO:12. A nucleic acid encoding pT1R3, or fragments thereof, may be amplified from porcine genomic DNA, mRNA or cDNA template using a forward primer selected from the group consisting of SEQ ID NO:3, SEQ ID NO:7, and a reverse primer selected from the group consisting of SEQ ID NO:4, SEQ ID NO:8 and SEQ ID NO:9. Oligonucleotides can be prepared by any suitable method, including chemical synthesis. Oligonucleotides can be synthesized using commercially available reagents and instruments. Alternatively, they can be purchased through commercial sources. Methods of synthesizing oligonucleotides are well known in the art (see, e.g, Narang et al., Meth. Enzymol. 68:90-99, 1979; Brown et al., Meth. Enzymol. 68:109-151, 1979; Beaucage et al., Tetrahedron Lett. 22:1859-1862, 1981; and the solid support method of U.S. Pat. No. 4,458,066).

A. PCR Identification of pT1R Alleles

In some embodiments, PCR is used to amplify nucleic acids encoding pT1R1 and/or pT1R3. A general overview of the applicable technology can be found in PCR Protocols: A Guide to Methods and Applications (Innis et al. eds. (1990)) and PCR Technology: Principles and Applications for DNA Amplification (Erlich, ed. (1992)). In addition, amplification technology is described in U.S. Pat. Nos. 4,683,195 and 4,683,202.

PCR permits the copying, and resultant amplification of a target nucleic acid, e.g., a nucleic acid encoding pT1R1 or pT1R3. Briefly, a target nucleic acid, e.g. DNA from a sample from a pocine, is combined with a sense and antisense primers, dNTPs, DNA polymerase and other reaction components. (See, Innis et al., supra) The sense primer can anneal to the antisense strand of a DNA sequence of interest. The antisense primer can anneal to the sense strand of the DNA sequence, downstream of the location where the sense primer anneals to the DNA target. In the first round of amplification, the DNA polymerase extends the antisense and sense primers that are annealed to the target nucleic acid. The first strands are synthesized as long strands of indiscriminate length. In the second round of amplification, the antisense and sense primers anneal to the parent target nucleic acid and to the complementary sequences on the long strands. The DNA polymerase then extends the annealed primers to form strands of discrete length that are complementary to each other. The subsequent rounds serve to predominantly amplify the DNA molecules of the discrete length.

In some embodiments, the isolated pT1R1 is encoded by a nucleic acid amplified from porcine genomic DNA, mRNA or cDNA template using a forward primer selected from the group consisting of SEQ ID NO:5, SEQ ID NO:10 and SEQ ID NO:11, and a reverse primer selected from the group consisting of SEQ ID NO:6 and SEQ ID NO:12. In some embodiments, the isolated pT1R3 is encoded by a nucleic acid amplified from porcine genomic DNA, mRNA or cDNA template using a forward primer selected from the group consisting of SEQ ID NO:3, SEQ ID NO:7, and a reverse primer selected from the group consisting of SEQ ID NO:4, SEQ ID NO:8 and SEQ ID NO:9.

B. Detection of amplified products

Amplified products can be detected using any means known in the art, including, e.g., denaturing gel electrophoresis (see, e.g., Erlich, ed., PCR TECHNOLOGY, PRINCIPLES AND APPLICATIONS FOR DNA AMPLIFICATION, W. H. Freeman and Co, New York, 1992, Chapter 7), direct sequencing, and HPLC-based analysis. Suitable sequence methods include e.g., dideoxy sequencing-based methods and Maxam and Gilbert sequence (see, e.g., Sambrook and Russell, supra). Suitable HPLC-based analyses include, e.g., denaturing HPLC (dHPLC) as described in e.g., Premstaller and Oefner, LC-GC Europe 1-9 (July 2002); Bennet et al., BMC Genetics 2:17 (2001); Schrimi et al., Biotechniques 28(4):740 (2000); and Nairz et al., PNAS USA 99(16):10575-10580 (2002); and ion-pair reversed phase HPLC-electrospray ionization mass spectrometry (ICEMS) as described in e.g., Oberacher et al.; Hum. Mutat. 21(1):86 (2003). Methods for characterizing single base changes in pT1R1 or pT1R3 include, e.g., single base extensions (see, e.g., Kobayashi et al, Mol. Cell. Probes, 9:175-182, 1995); single-strand conformation polymorphism analysis, as described, e.g, in Orita et al., Proc. Nat. Acad. Sci. 86, 2766-2770 (1989), allele specific oligonucleotide hybridization (ASO) (e.g., Stoneking et al., Am. J. Hum. Genet. 48:70-382, 1991; Saiki et al., Nature 324, 163-166, 1986; EP 235,726; and WO 89/11548); and sequence-specific amplification or primer extension methods as described in, for example, WO 93/22456; U.S. Pat. Nos. 5,137,806; 5,595,890; 5,639,611; and U.S. Pat. No. 4,851,331; 5′-nuclease assays, as described in U.S. Pat. Nos. 5,210,015; 5,487,972; and 5,804,375; and Holland et al., 1988, Proc. Natl. Acad. Sci. USA 88:7276-7280.

Although the methods typically employ PCR steps, other amplification protocols may also be used. Suitable amplification methods include ligase chain reaction (see, e.g., Wu & Wallace, Genomics 4:560-569, 1988); strand displacement assay (see, e.g., Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86:1173-1177, 1989); and self-sustained sequence replication (3SR) (Guatelli et al., Proc. Natl. Acad. Sci. USA 87:1874-1878, 1990; WO 92/08800). Alternatively, methods that amplify the probe to detectable levels can be used, such as Qβ-replicase amplification (Kramer & Lizardi, Nature 339:401-402, 1989; Lomeli et al., Clin. Chem. 35:1826-1831, 1989). A review of known amplification methods is provided, for example, by Abramson and Myers in Current Opinion in Biotechnology 4:41-47, 1993.

### V. Immunological Detection of pT1R Polypeptides

In addition to the detection of pT1R1 and pT1R3 genes and gene expression using nucleic acid hybridization technology, one can also use immunoassays to detect pT1R1- and pT1R3-comprising umami taste receptors of the invention. Such assays are useful for screening for modulators of pT1R1- and pT1R3-comprising umami taste receptors, as well as for therapeutic and diagnostic applications. Immunoassays can be used to qualitatively or quantitatively analyze pT1R1- and pT1R3-comprising umami taste receptors. A general overview of the applicable technology can be found in Harlow & Lane, Antibodies: A Laboratory Manual (1988).

Methods of producing polyclonal and monoclonal antibodies that specifically bind monomers, heterodimers, and homodimers comprising pT1R1 and/or pT1R3, or immunogenic fragments thereof, are known to those of skill in the art (see, e.g., Coligan, Current Protocols in Immunology (1991); Harlow & Lane, supra; Goding, Monoclonal Antibodies: Principles and Practice (2d ed. 1986); Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of immunogens comprising portions of pT1R1 or pT1R3 may be used to produce antibodies specifically reactive with pT1R1 or pT1R3 or homologues thereof. For example, recombinant pT1R1 or pT1R3 polypeptide (encoded by a sequence comprising SEQ ID NOS:1 and 2, respectively) or antigenic fragment thereof, can be isolated as described herein. Recombinant protein can be expressed in eukaryotic or prokaryotic cells as described above, and purified as generally described above for use as an immunogen. Alternatively, a synthetic peptide derived from the sequences disclosed herein and conjugated to a carrier protein can be used an immunogen. Naturally occurring pT1R1 or pT1R3 protein may also be used either in pure or impure form.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

For example, polyclonal and monoclonal antibodies raised to T1R can be selected to obtain only those polyclonal and monoclonal antibodies that are specifically immunoreactive with pT1R and pT1R3 (e.g., a pT1R1 encoded by SEQ ID NO:1 or a pT1R3 encoded by SEQ ID NO:2) and not with T1R from other species. This selection may be achieved by subtracting out antibodies that cross-react with molecules such as T1R from other species. In addition, polyclonal and monoclonal antibodies raised to T1R polymorphic variants, alleles, orthologs, and conservatively modified variants can be selected to obtain only those antibodies that recognize specific fragments of pT1R1 or pT1R3. For example, polyclonal or monoclonal antibodies raised can be selected for only those antibodies that recognize T1R1 polypeptides encoded by a nucleic acid amplified from porcine genomic DNA, mRNA or cDNA template using a forward primer selected from the group consisting of SEQ ID NO:5, SEQ ID NO:10 and SEQ ID NO:11, and a reverse primer selected from the group consisting of SEQ ID NO:6 and SEQ ID NO:12, but not other pT1R1 fragments. Similarly, polyclonal or monoclonal antibodies raised can be selected for only those antibodies that recognize T1R3 polypeptides encoded by a nucleic acid amplified from porcine genomic DNA, mRNA or cDNA template using a forward primer selected from the group consisting of SEQ ID NO:3, SEQ ID NO:7, and a reverse primer selected from the group consisting of SEQ ID NO:4, SEQ ID NO:8 and SEQ ID NO:9, but not other pT1R3 fragments. A variety of immunoassay formats may be used to select antibodies specifically immunoreactive with pT1R1 and/or pT1R3. For example, solid-phase ELISA immunoassays are routinely used to select antibodies specifically immunoreactive with a protein (see, e.g., Harlow and Lane, Antibodies: A Laboratory Manual (1988), and Harlow and Lane, Using Antibodies: A Laboratory Manual (1998), Cold Spring Harbor Laboratory Press, for descriptions of immunoassay formats and conditions that can be used to determine specific immunoreactivity). Typically a specific or selective reaction will be at least twice background signal or noise and more typically more than 10 to 100 times background.

Immunoassays known in the art can be used to assess the binding specificity and binding affinity of antibodies that specifically bind to pT1R1 or pT1R3. Typically, polyclonal antisera with a titer of 10⁴ or greater are selected and tested for their cross reactivity against non-pT1R1 or non-pT1R3 proteins, using a competitive binding immunoassay. Specific polyclonal antisera and monoclonal antibodies will usually bind with a Kd of at least about 0.1 mM, more usually at least about 1 μM, preferably at least about 0.1 μM or better, and most preferably, 0.01 μM or better. Antibodies specific only for pT1R1 or pT1R3, e.g., encoded by a sequence comprising SEQ ID NOS:1 or 2, respectively can also be made, by subtracting out other cross-reacting homologues from a species such as a human or non-human mammal. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Ten, eds., 7th ed. 1991). Immunoassays for detecting pT1R1 or pT1R3 or immunogenic fragments thereof in samples may be either competitive or noncompetitive. Noncompetitive immunoassays are assays in which the amount of antigen is directly measured. In competitive assays, the amount of the pT1R1 or pT1R3 present in the sample is measured indirectly by measuring the amount of known, added (exogenous) pT1R1 or pT1R3 displaced (competed away) from an anti-pT1R1 or anti-pT1R3 antibody by the unknown pT1R1 or pT1R3 present in a sample. Western blot (immunoblot) analysis can also be used to detect and quantify the presence of the pT1R1 or pT1R3 polypeptides in the sample.

### VI. Assays for modulators

One embodiment of the invention provides methods to identify modulators (e.g., activators, inhibitors, stimulators, enhancers, agonists, and antagonists) of porcine umami taste receptors, e.g., porcine umami taste receptors comprising pT1R1 and pT1R3 monomers, heterodimers, and homodimers. Such modulators of umami taste transduction are useful for pharmacological and genetic modulation of umami taste signaling pathways, and for the discovery of novel umami taste ligands. These methods of screening can be used to identify high affinity agonists and antagonists of umami taste cell activity. These modulatory compounds can then be used in the animal feed and veterinaray pharmaceutical industries to customize taste, for example to increase the food appeal of animal feed, animal feed supplements or animal medications. Thus, the invention provides assays for taste modulation, where the receptor comprising a pT1R1 or pT1R3 monomer, heterodimer, or homodimer acts as a direct or indirect reporter molecule for the effect of modulators on umami taste transduction.

The activity of pT1R1 and pT1R3 monomers, homodimers, and heterodimers can be assessed using a variety of in vitro and in vivo assays that determine functional, physical and chemical effects, e.g., measuring ligand binding (e.g., by radioactive ligand binding), second messengers (e.g., cAMP, cGMP, IP₃, DAG, or Ca²⁺), ion flux, phosphorylation levels, transcription levels, neurotransmitter levels, and the like. Furthermore, such assays can be used to test for inhibitors and activators of pT1R1 and pT1R3 monomers, homodimers, and heterodimers. In some embodiments, the assays employ genetically altered versions of pT1R1 and pT1R3 monomers, homodimers, and heterodimers. Such modulators of taste transduction activity are useful for customizing taste, e.g. to increase the palatability or food appeal of animal feeds, supplements, and medicaments.

The pT1R1 and pT1R3 monomers, homodimers, and heterodimers used in the assays can be selected from a polypeptide encoded by SEQ ID NO:1, SEQ ID NO:2 or subsequence or conservatively modified variant thereof. Alternatively, the pT1R1 and pT1R3 monomers, homodimers, and heterodimers of the assay can be derived from a eukaryote and include an amino acid subsequence having amino acid sequence identity to a polypeptide encoded by a nucleic acid sharing at least about 90%, 91%, 92%, 93%, 94%, 95%, 96%, 97%, 98%, 99% sequence identity to SEQ ID NO:1 or SEQ ID NO:2, or a subsequence or conservatively modified variant thereof. Generally, the amino acid sequence identity of the T1R1 and T1R3 will be at least about 90%, 91%, 92%, 93%, 94%, 95%, 96%, 97%, 98%, 99% to SEQ ID NO:13 or 14, respectively.

In some embodiments, the pT1R1 used in the assays is encoded by a nucleic acid amplified from porcine genomic DNA, mRNA or cDNA template using a forward primer selected from the group consisting of SEQ ID NO:5, SEQ ID NO:10 and SEQ ID NO:11, and a reverse primer selected from the group consisting of SEQ ID NO:6 and SEQ ID NO:12. In some embodiments, the pT1R3 used in the assays is encoded by a nucleic acid amplified from porcine genomic DNA, mRNA or cDNA template using a forward primer selected from the group consisting of SEQ ID NO:3, SEQ ID NO:7, and a reverse primer selected from the group consisting of SEQ ID NO:4, SEQ ID NO:8 and SEQ ID NO:9.

Optionally, the polypeptide of the assays will comprise a domain of pT1R1 or pT1R3, such as an extracellular domain, transmembrane domain, cytoplasmic domain, ligand binding domain, subunit association domain, active site, and the like. Either pT1R1 or pT1R3, or a domain thereof, can be covalently linked to a heterologous protein to create a chimeric protein used in the assays described herein. For example, ligand binding assays can be performed using one or more extracellular domains of the pT1R1, and optionally including one or more extracellular domains of the pT1R3 polypeptide. In another embodiments, ligand binding assays can be performed using pT1R1, and optionally pT1R3 polypeptides wherein the cytoplasmic domains have been truncated.

Modulators of pT1R1 and pT1R3 monomers, homodimers, and heterodimers activity are tested using pT1R1 and pT1R3 polypeptides as described above, either recombinant or naturally occurring. The protein can be isolated, expressed in a cell, expressed in a membrane derived from a cell, expressed in tissue or in an animal, either recombinant or naturally occurring. For example, tongue slices, dissociated cells from a tongue, transformed cells, or membranes can be used. Modulation is tested using one of the in vitro or in vivo assays described herein. Taste transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and or cytoplasmic domain of a receptor. Furthermore, ligand-binding domains of the protein of interest can be used in vitro in soluble or solid state reactions to assay for ligand binding.

Ligand binding to pT1R1 and pT1R3 monomers, homodimers, and heterodimers, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index) hydrodynamic (e.g., shape), chromatographic, or solubility properties.

In some embodiments, ligand binding to pT1R1 and pT1R3 monomers, homodimers, and heterodimers, a domain, or chimeric protein can be tested in an intact host cell. In some embodiments, a nucleic acid encoding a pT1R1 subunit is transfected and expressed in the host cell. In a related embodiment, nucleic acids encoding a pT1R1 subunit and a pT1R3 subunit are transfected and expressed in the host cell. In some embodiments, nucleic acids encoding a pT1R1 subunit and a G protein alpha subunit are transfected and expressed in the host cell. The transfected host cell is then contacted with test modulators of the expressed pT1R receptor, and a physical (e.g., ligand binding) or functional effect (e.g., second messenger changes, e.g., increases or decreases in cytoplasmic cAMP, cGMP, IP₃, DAG, or Ca²⁺) is determined. In some embodiments, the determination of the physical or functional effect of the test modulator is compared to the physical or functional effect of a control host cell that is not contacted with a test modulator. In one embodiment, the screening methods determine increases or decreases of Ca²⁺ released into the cytoplasm of a host cell expressing a pT1R1 (and optionally a pT1R3 and/or a G protein alpha subunit) and contacted with a test modulator in comparison to a control host cell expressing a pT1R1 (and optionally a pT1R3 and/or a G protein alpha subunit) and not contacted with a test modulator.

Receptor-G protein interactions can also be examined. For example, binding of the G protein to the receptor or its release from the receptor can be examined. For example, in the absence of GTP, an activator will lead to the formation of a tight complex of a G protein (all three subunits) with the receptor. This complex can be detected in a variety of ways, as noted above. Such an assay can be modified to search for inhibitors. Add an activator to the receptor and G protein in the absence of GTP, form a tight complex, and then screen for inhibitors by looking at dissociation of the receptor-G protein complex. In the presence of GTP, release of the alpha subunit of the G protein from the other two G protein subunits serves as a criterion of activation.

An activated or inhibited G protein will in turn alter the properties of target enzymes, channels, and other effector proteins. The classic examples are the activation of cGMP phosphodiesterase by transducin in the visual system, adenylate cyclase by the stimulatory G protein, phospholipase C by Gq and other cognate G proteins, and modulation of diverse channels by Gi and other G proteins. Downstream consequences can also be examined such as generation of diacyl glycerol and IP3 by phospholipase C, and in turn, for calcium mobilization by IP3.

Activated GPCR receptors become substrates for kinases that phosphorylate the C-terminal tail of the receptor (and possibly other sites as well). Thus, activators will promote the transfer of ³²P from gamma-labeled GTP to the receptor, which can be assayed with a scintillation counter. The phosphorylation of the C-terminal tail will promote the binding of arrestin-like proteins and will interfere with the binding of G proteins. The kinase/arrestin pathway plays a key role in the desensitization of many GPCR receptors. For example, compounds that modulate the duration a taste receptor stays active would be useful as a means of prolonging a desired taste or cutting off an unpleasant one. For a general review of GPCR signal transduction and methods of assaying signal transduction, see, e.g., Methods in Enzymology, vols. 237 and 238 (1994) and volume 96 (1983); Bourne et al., Nature 10:349:117-27 (1991); Bourne et al., Nature 348:125-32 (1990); Pitcher et al., Annu Rev. Biochem. 67:653-92 (1998).

Samples or assays that are treated with a potential pT1R1 and pT1R3 monomer, homodimer, or heterodimer inhibitor or activator are compared to control samples without the test compound, to examine the extent of modulation. Control samples (untreated with activators or inhibitors) are assigned a relative pT1R1 or pT1R3 activity value of 100. Inhibition of pT1R1 or pT1R3 is achieved when the pT1R1 or pT1R3 activity value relative to the control is about 90%, optionally 50%, optionally 25-0%. Activation of pT1R1 or pT1R3 is achieved when the pT1R1 or pT1R3 activity value relative to the control is 110%, optionally 150%, 200-500%, or 1000-2000%.

Changes in ion flux may be assessed by determining changes in polarization (i.e., electrical potential) of the cell or membrane expressing a pT1R1 or pT1R3 monomer, homodimer, or heterodimer. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., PFlugers. Archiv. 391:85 (1981). Other known assays include: radiolabeled ion flux assays and fluorescence assays using voltage-sensitive dyes (see, e.g., Vestergarrd-Bogind et al., J. Membrane Biol. 88:67-75 (1988); Gonzales & Tsien, Chem. Biol. 4:269-277 (1997); Daniel et al., J. Pharmacol. Meth. 25:185-193 (1991); Holevinsky et al., J. Membrane Biology 137:59-70 (1994)). Generally, the compounds to be tested are present in the range from 1 μM to 100 mM.

The effects of the test compounds upon the function of the polypeptides can be measured by examining any of the parameters described above. Any suitable physiological change that affects GPCR activity can be used to assess the influence of a test compound on the polypeptides of this invention. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP₃ or cAMP.

Assays for G protein coupled receptors include cells that are loaded with ion or voltage sensitive dyes to report receptor activity. Assays for determining activity of such receptors can also use known agonists and antagonists for other G protein coupled receptors as negative or positive controls to assess activity of tested compounds. In assays for identifying modulatory compounds (e.g., agonists, antagonists), changes in the level of ions in the cytoplasm or membrane voltage will be monitored using an ion-sensitive or membrane voltage fluorescent indicator, respectively. Among the ion-sensitive indicators and voltage probes that may be employed are those disclosed in the Molecular Probes 1997 Catalog. For G protein coupled receptors, promiscuous G proteins such as G.alpha.15 and G.alpha.16 can be used in the assay of choice (Wilkie et al., PNAS USA 88:10049-10053 (1991)). Such promiscuous G proteins allow coupling of a wide range of receptors.

Receptor activation typically initiates subsequent intracellular events, e.g., increases in second messengers such as inositol triphosphate (IP₃), which releases intracellular stores of calcium ions. Activation of some G protein coupled receptors stimulates the formation of IP₃ through phospholipase C-mediated hydrolysis of phosphatidylinositol (Berridge & Irvine, Nature 312:315-21 (1984)). IP₃ in turn stimulates the release of intracellular calcium ion stores. Thus, a change in cytoplasmic calcium ion levels, or a change in second messenger levels such as IP₃ can be used to assess G protein coupled receptor function. Cells expressing such G protein coupled receptors may exhibit increased cytoplasmic calcium levels as a result of contribution from both intracellular stores and via activation of ion channels, in which case it may be desirable although not necessary to conduct such assays in calcium-free buffer, optionally supplemented with a chelating agent such as EGTA, to distinguish fluorescence response resulting from calcium release from internal stores.

Other assays can involve determining the activity of receptors which, when activated, result in a change in the level of intracellular cyclic nucleotides, e.g., cAMP or cGMP, by activating or inhibiting enzymes such as adenylate cyclase. There are cyclic nucleotide-gated ion channels, e.g., rod photoreceptor cell channels and olfactory neuron channels that are permeable to cations upon activation by binding of cAMP or cGMP (see, e.g., Altenhofen et al., PNAS USA. 88:9868-9872 (1991) and Dhallan et al., Nature 347:184-187 (1990)). In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. Cells for this type of assay can be made by co-transfection of a host cell with DNA encoding a cyclic nucleotide-crated ion channel, GPCR phosphatase and DNA encoding a receptor (e.g., certain glutamate receptors, muscarinic acetylcholine receptors, dopamine receptors, serotonin receptors, and the like), which, when activated, causes a change in cyclic nucleotide levels in the cytoplasm.

In one embodiment, the changes in intracellular cAMP or cGMP can be measured using immunoassays. The method described in Offermanns & Simon, J. Biol. Chem. 270:15175-15180 (1995) may be used to determine the level of cAMP. Also, the method described in Felley-Bosco et al., Am. J. Resp. Cell and Mol. Biol. 11:159-164 (1994) may be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist to cpm in the presence of buffer control. Likewise, fold inhibition is determined by calculating the ratio of cpm in the presence of antagonist to cpm in the presence of buffer control (which may or may not contain an agonist).

In another embodiment, transcription levels can be measured to assess the effects of a test compound on signal transduction. A host cell containing the protein of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, firefly luciferase, bacterial luciferase, β-galactosidase and alkaline phosphatase. Furthermore, the protein of interest can be used as an indirect reporter via attachment to a second reporter such as green fluorescent protein (see, e.g., Mistili & Spector, Nature Biotechnology 15:961-964 (1997)).

The amount of transcription is then compared to the amount of transcription in either the same cell in the absence of the test compound, or it may be compared with the amount of transcription in a substantially identical cell that lacks the protein of interest. A substantially identical cell may be derived from the same cells from which the recombinant cell was prepared but which had not been modified by introduction of heterologous DNA. Any difference in the amount of transcription indicates that the test compound has in some manner altered the activity of the protein of interest.

A. Modulators

The compounds tested as modulators of pT1R1 or pT1R3 monomers, homodimers, or heterodimers can be any small chemical compound, or a biological entity, including without limitation, a protein, a sugar, a nucleic acid or a lipid. Alternatively, modulators can be genetically altered versions of pT1R1 or pT1R3 monomers, homodimers, or heterodimers (e.g., extracellular domains or soluble forms of pT1R1 or pT1R3). Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Additional compounds that can be used include peptides of Formula I:

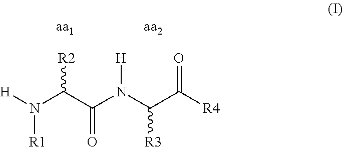

wherein R₁═H, CH₃CO; aaᵢ and aa₂ are independently selected from any one of the 20 naturally occurring amino acids; R4=OH, NH₂, NH₂R₅, OMe, OPr, OMe, OPr ro aa3, wherein aa3=L-Lys, L-Glu, Gly, L-Ala, L-Asn, L-Gln, L-Hyp, L-Ser, L-Thr, L-Asp, L-Amp, D-Ala, D-Asn, D-Gln, D-Phe, D-Ser, D-Thr, D-Trp, D-Asp, or D-Glu; and R₅=Me, Et, Pr, Ph.

Additional compounds that can be used include aromatics, heteroaromatics, oxalamides, acrylamides, esters and polyenamides as set forth in U.S. Patent Publication No. 20050197387; compounds set forth in U.S. Patent Publication Nos. 20040202619; 20040202760; 20060057268; 20060068071; and 2006045953; and PCT Publication No. WO2005041684.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

B. Solid State and Soluble High throughput Assays

In one embodiment the invention provides soluble assays using molecules such as a domain such as ligand binding domain, an extracellular domain, a transmembrane domain (e.g., one comprising seven transmembrane regions and cytosolic loops), the transmembrane domain and a cytoplasmic domain, an active site, a subunit association region, etc.; a domain that is covalently linked to a heterologous protein to create a chimeric molecule comprising a pT1R1, pT1R3 monomer, homodimer, or heterodimer; or a cell or tissue expressing pT1R1, pT1R3 monomer, homodimer, or heterodimer, either naturally occurring or recombinant. In another embodiment, the invention provides solid phase based in vitro assays in a high throughput format, where the domain, chimeric molecule, pT1R1, pT1R3 monomer, homodimer, or heterodimer, or cell or tissue expressing pT1R1, pT1R3 monomer, homodimer, or heterodimer is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay several different plates per day; assay screens for up to about 6,000-20,000 different compounds is possible using the integrated systems of the invention. More recently, microfluidic approaches to reagent manipulation have been developed, e.g., by Caliper Technologies (Palo Alto, Calif.).

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest (e.g., the taste transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.)

Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids (SEQ ID NO:33). Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

C. Computer-Based Assays

Yet another assay for compounds that modulate pT1R1 or pT1R3 monomer, homodimer, or heterodimer activity involves computer assisted drug design, in which a computer system is used to generate a three-dimensional structure of a pT1R1 or pT1R3 monomer, homodimer, or heterodimer based on the structural information encoded by the amino acid sequence. The input amino acid sequence interacts directly and actively with a preestablished algorithm in a computer program to yield secondary, tertiary, and quaternary structural models of the protein. The models of the protein structure are then examined to identify regions of the structure that have the ability to bind, e.g., ligands. These regions are then used to identify ligands that bind to the protein.

The three-dimensional structural model of the protein is generated by entering protein amino acid sequences of at least 10 amino acid residues or corresponding nucleic acid sequences encoding a pT1R1 or pT1R3 polypeptide into the computer system. The amino acid sequence of the polypeptide of the nucleic acid encoding the polypeptide is selected from the group consisting of SEQ ID NO:1 or SEQ ID NO:2 and conservatively modified versions thereof. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the pT1R1, pT1R3 monomer, homodimer, or heterodimer protein to identify ligands that bind to pT1R1, pT1R3 monomer, homodimer, or heterodimer. Binding affinity between the protein and ligands is determined using energy terms to determine which ligands have an enhanced probability of binding to the protein.

Computer systems are also used to screen for mutations, polymorphic variants, and alleles of pTAS1R1 and pTAS1R3 genes. Such mutations can be associated with disease states or genetic traits. As described above, GENECHIP™. and related technology can also be used to screen for mutations, polymorphic variants, alleles and interspecies homologs. Once the variants are identified, diagnostic assays can be used to identify patients having such mutated genes. Identification of the mutated pTAS1R1 and pTAS1R3 genes involves receiving input of a first nucleic acid or amino acid sequence encoding pTAS1R1 and pTAS1R3, selected from the group consisting of SEQ ID NO:1 and SEQ ID NO:2 and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in pTAS1R1 and pTAS1R3 genes, and mutations associated with disease states and genetic traits.

### VII. Kits

pT1R1 and pT1R3 and their variants and alleles are useful tools for more specific and sensitive identification of porcines expressing umami taste receptors, i.e., to select porcines for breeding or for identification of porcines as animals to test umami enhancing compounds.

The invention also provides kits and solutions for detecting the pT1R1 and pT1R3 polypeptides and nucleic acids described herein. For example, the invention provides kits that include one or more reaction vessels that have aliquots of some or all of the reaction components of the invention in them. Aliquots can be in liquid or dried form. Reaction vessels can include sample processing cartridges or other vessels that allow for the containment, processing and/or amplification of samples in the same vessel. Such kits allow for ready detection of amplification products of the invention into standard or portable amplification devices. The kits can also include written instructions for the use of the kit to amplify and control for amplification of pT1R1 and pT1R3 nucleic acids.

Kits can include, for instance, amplification reagents comprising primers sufficient to amplify pT1R1 and pT1R3 (e.g., SEQ ID NOS: 3 and 4, 5 and 6, 7 and 8, 9 and 10, or 11 and 12) and at least one probe for amplifying and detecting the polynucleotide sequence. In addition, the kit can include nucleotides (e.g., A, C, G and T), a DNA polymerase and appropriate buffers, salts and other reagents to facilitate amplification reactions.

### EXAMPLES

The following examples are illustrative and non limiting examples of the experiments performed for the identification, isolation and characterization of the porcine umami receptor.

### Example 1

### RNA Isolation and cDNA Preparation

Vallate papilla tissue samples were obtained from a 6-month-old male pig. The total RNA was extracted using RNeasy fibrous tissue mini kit. (Qiagen 74704). Concentration and quality of RNA were determinated by optical density measurement and agarose gel electrophoresis. 3 μg of RNA was subsequently reverse transcribed using random hexanucleotide primers and SuperScript III enzyme all part of GeneRacer kit (Invitrogen L1500-01).

Taste Receptor Family 1 form 3 (T1R3): A porcine expressed sequence tag (EST) with high homology to human T1R3 was located in a public domain library (pig ESTs database from Iowa State University). The RACE PCR technique was used to obtain cDNA for adjacent 3′ and 5′ regions of the T1R3 sequence using mRNA prepared from taste bud papillae of a 6 month-old male pig. Candidate PCR products were sequenced and the RACE process was repeated until the full length mRNA sequence was determined.

Before starting the RNA ligase-mediated rapid amplification of 5′ and 3′ cDNA ends (RLM-RACE) gene specific primers (TSR3-GSP) were made from a 200 bp pig EST sequence found in the NCBI database by blasting human and mouse TAS1R3 published sequences. The temperature cycles for the set of primers were as follows: 30 sec at 95° C., 1 min at 55° C., and 1 min at 72° C. These cycles were carried out 40 times.

Taste Receptor Family 1 form 1 (T1R1): To obtain the pig T1R1 receptor sequence, degenerate PCR primers (DP) were designed that covered areas of high homology to the mouse, human, and/or cat genes. Primer pairs were used to amplify pig mRNA using relaxed PCR conditions. Eventually primer sets were found that amplified a portion of the T1R1 as verified by sequencing of the PCR product. The RACE PCR technique was used to obtain cDNA for the rest of the T1R1 sequence using mRNA from pig taste buds. Candidate PCR products were sequenced and the RACE process was repeated until the full length mRNA sequence was determined.

Eight primer pairs were designed using Consensus-Degenerate Hybrid Oligonucleotide Primers (CODEHOP) strategy. Optimal primers were made to the pig predicted sequence from BlockMaker generated alignments of multiple known TAS1R1 protein sequences. One degenerate primer combination (15 and 17) amplified a 140 bp DNA sequence with high homology to the human and mouse T1R1. The degenerate PCR conditions were 94° C. for 30 sec, 65° C. for 1 min, and 68° C. for 2 min for 40 cycles.

### Example 2

### Pig T1R3 and T1R1 Full-Length cDNA Amplification and Cloning

Full length products were amplified by PCR using Platinum Taq DNA Polymerase High Fidelity (Invitrogen 11304-011) and cloned into pCR8/GW/TOPO TA vector (Invitrogen K2500-20). Pig T1R3 was amplified by 2 step PCR and nested PCR using the following conditions: 94° C. for 30 sec and 68° C. for 2 min 30 sec repeated 30 times. Pig T1R1 was also amplified by 2 step PCR and nested PCR using the same conditions. Both sequences were then verified by automated sequencing performed on ABI3730 sequencer. Next, the open reading frame was recombined into pcDNA6.2/V5-DEST Gateway vector (Invitrogen 12489-027) using Gateway BP clonase II enzyme mix (Invitrogen 11789-020) for expression in mammalian cells.

Oligonucleotide primers used in PCRs:
T1R3 GSP forward:
(SEQ ID NO: 3)
5′-ACTGCCGCGTGCACTCCTG-3′
T1R3 GSP reverse:
(SEQ ID NO: 4)
5′-GGAGGCCACAGGCACGTTG-3′
T1R1 DP forward 15:
(SEQ ID NO: 5)
5′-GGCAGTACCCCTCCTTCCTGMGNACNATHCC-3′
T1R1 DP reverse 17:
(SEQ ID NO: 6)
5′-CCTGCACGCCCAGCTGNCCRTARTC-3′
T1R3 forward:
(SEQ ID NO: 7)
5′-ATGCCAGGCCTGACTCTCCTG-3′
T1R3 reverse:
(SEQ ID NO: 8)
5′-TCACTGTGGGGTCAAGGGTGG-3′
T1R3 nested reverse:
(SEQ ID NO: 9)
5′-TCAGGGCACAGCCTGCTGGT-3′
T1R1 forward:
(SEQ ID NO: 10)
5′-ATGCCAGGCTCAGGGCGG-3′
T1R1 nested forward:
(SEQ ID NO: 11)
5′-ATGTTGCTCTGGGAGGCGTG-3′
T1R1 reverse:
(SEQ ID NO: 12)
5′-TCAGGTGGAGCCGCAGCG-3′

### Example 3

### Screening Assay for Ligands

Supplies and reagents: Lipofectamine 2000 (Invitrogen, 11668-027), CHO-K1 cell line (ATCC, CCL-61), 96 well flat bottom black plate (Costar 3603), Fluo-4 NW calcium assay kit (Molecular probes, F36205). Complete medium (CM): DMEM (GIBCO, 11960-044) supplemented with 10% FBS, 1X (P/S, sodium pyruvate, 25 mM HEPES).

CHO-K1 cells (American Type Culture) were seeded in 6-well-plate (˜500,000 cells per well) in 2 ml of CM and grown overnight at 37° C. with 5% CO₂. The next day a 0.5 ml transfection mixture was added to CM. The transfection mixture consisted of 0.25 ml of CM minus serum and antibiotics containing 10 μl of Lipofectamine plus 0.25 ml of the same medium containing both full-length pig taste receptors and mouse G alpha 15 G protein sub-unit DNA constructs. The transfection mixture was incubated at room temperature for 20 minutes before addition to cell cultures. The DNA expression constructs were generated in pcDNA6.2/V5-DEST (Invitrogen). The transfected cells were incubated for 24 hours.

For the umami taste assay, 50,000 transfected cells in 100 μl of media containing physiologic amount of L-glutamax were seeded in wells of a black 96-well-plate and incubated for an additional 24 hours.

The day of the assay, medium was replaced in all the wells by 100 μl of dye loading solution and incubated for 30 minutes at 37° C. and then for an additional 30 minutes at room temperature and protected from light. The plate was placed in FLx800 fluorescence reader set to excite at 484 nm and measure emission at 528 nm. The dye loading solution was removed and replaced with 100 μlassay buffer (HBSS with 0.2M HEPES buffer) containing test compounds. Each test compound was tested in triplicate wells. Fluorescence was read within 60 to 90 seconds at a sensitivity level of 75 to 85.

Any ligand that can be added to tissue culture media at levels that are not cytotoxic can be tested. An example test run is shown in Table 1. In this experiment, all treatments were replicated in 8 wells of a 96 well plate. The coefficient of variation for the amino acid treatments averaged 6.3% at 610 nm and 7.1% at 530 nm. The background fluorescence was defined as the signal at 460 and 530 nm when no treatment was added to the cells and was defined as a response ratio of 1.0. The positive control, thapsigargin gave a response index of 17.89. This compound triggers a maximum response because it directly delivers calcium to the cytoplasm and bypasses receptor-related events. Among amino acids, alanine gave the largest response index (3.47) while cysteine did not give a response (0.83).

This assay system identified molecules that stimulate the umami receptor and cause a physiological response (calcium release into the cytoplasm) that mimics the response in a normal taste cell. The results are summarized in Table 1 below.

TABLE 1
Effect of treatments on calcium influx via Pig umami receptor

| | Index |
|---|---|
| Absorbance | Relative |

| Treatment | Mean | SD | to control |
|---|---|---|---|
| Aspartate 1 mM | 8619 | 495 | 1.03 |
| Aspartate 10 mM | 10352 | 572 | 1.24¹ |
| Serine 5 mM | 8372 | 307 | 1.00 |
| Serine 10 mM | 9697 | 360 | 1.16 |
| Glutamine 1 mM | 10901 | 345 | 1.31¹ |
| Glutamine 10 mM | 10038 | 496 | 1.20¹ |
| Glycine 1 mM | 8874 | 359 | 1.06 |
| Glycine 5 mM | 9543 | 474 | 1.14 |
| L-AP4 10 mM | 9316 | 455 | 1.12 |
| L-AP4 5 mM | 8907 | 593 | 1.07 |
| Lysine HCl 10 mM | 9319 | 1041 | 1.12 |
| Lysine HCl 30 mM | 9675 | 624 | 1.16 |
| Leucine 10 mM | 9163 | 341 | 1.10 |
| Leucine 30 mM | 10161 | 254 | 1.22¹ |
| IMP 2.5 mM | 9161 | 100 | 1.10 |
| Control (no treatment) | 8340 | 440 | 1.00 |

¹Significantly different from control (1.0).

Data are ratio of calcium influx due to treatment relative to influx due to control (no treatment). Mean values are from three replicate wells. Most amino acids gave a weak and marginally significant signal, especially at higher concentrations. Aspartate, glutamine and leucine gave the strongest and the most significant signal.

### Example 4

### Preferences for Amino Acids by Weaned Piglets

This example tests preferences in drinking water for amino acids L-Glutamine and L-Lysine in weaned piglets maintained in ad libitum conditions.

### Materials and Methods

A total number of 58 pigs (Landrace×Large White) of 18±2 Kg BW, half males and half females, were used in this study. The test pigs were selected according to their level of stress and behaviour in front of social isolation, reaction to novelty and training to perform a choice between two containers that had the test solution (sugar at 0.5M) and control solution (tap water) after an habituation period of four days. During the habituation period, individual pigs were maintained in contiguous pens in front of two unfamiliar red plastic containers (double-choice test) during 10 minutes every day. On the fourth day, animals were trained for performing the test. Animals that showed clear signs of distress and did not perform the trial this day were eliminated from test group. Selected test animals were maintained in groups of four and three pen mates under ad libitum conditions, feed and water, with a balanced diet according to their requirements. During the experimental sessions, pigs were exposed simultaneously to two different solutions (approximately 250 g in each container) for a time period of two minutes. One of the solutions was tap water (control) and the other contained a 0.5M solution of one of the amino acids tested (test). Volume depletion of both test and control solutions were recorded and the % preference related to the test solution calculated as follows:

$$\% preference = \frac{Test solution consumed ( g )}{Total solution consumed ( test + control ) ( g )} \times 100$$

### Results and Discussion

A total number of 29 test pigs were selected after the 4-day habituation period. Almost 50% of animals were properly trained using this methodology. Compared to a negative treatment (control vs. control) where preferences values are neutral (around 50%) preferences over tap water for L-glutamine were 60% and 79% for the 1st and 2nd studies respectively, while preferences for L-Lysine were only about 38%. The results are summarized in Table 2.

TABLE 2
% of preference for L-Glutamine or L-Lysine

| | Dose | Number of | Mean | |
|---|---|---|---|---|
| | (M) | pigs tested | (%) | SD |
| L-Glutamine | 0.5 | 18 | 59.56 | 26.14 |
| L-Glutamine | 0.5 | 29 | 79.28 | 26.35 |
| L-Lysine | 0.5 | 11 | 37.66 | 31.18 |

The “in vivo” data of this example confirm the “in vitro” data shown in Example 3 where L-Glutamine had a high degree of stimulation of the umami taste receptor (T1R1/T1R3) expressed in the CHO-K1 cell system. Similarly, the “in vivo” results of this example show a negative preference for L-Lysine that confirms as well the data of Example 3 showing little or no capacity of L-Lysine to stimulate the pig umami receptor.

### Example 5

### In silico Characterization of Pig T1R1 and T1R3

This example demonstrates the use of bioinformatic software to characterize domains, regions and amino acid residues contained within pig T1R1 and T1R3 proteins.

### Methods

Evaluation of the pig T1R1 and T1R3 nucleotide sequences. The pig T1R1 contains 2535 base pairs. The pig T1R1 sequence begins with a start codon (ATG) and ends with a stop codon (TGA), indicating that the entire pig T1R1 open reading frame is present. This was confirmed by determining the pig T1R1 open reading frame using Vector NTI 9.0 software.

The pig T1R3 nucleotide sequence contains 2568 base pairs. The pig T1R3 sequence begins with a start codon (ATG) and ends with a stop codon (TGA), indicating that the entire pig T1R3 open reading frame is present. This was confirmed by determining the pig T1R3 open reading frame using Vector NTI 9.0 software.

Nucleotide translation. The pig T1R1 and T1R3 nucleotide sequences were translated into amino acid sequence using the translate tool at Expasy (on the worldwide web at us.expasy.org/tools/dna.html). Since both T1R1 and T1R3 nucleotide sequences began with a start codon (ATG), the nucleotide sequence was translated in the first frame, meaning that ATG was designated as the first, or start, codon for translation. This also ensured that the protein sequence began with methionine, which is required for all mammalian proteins. The T1R1 protein contains 844 amino acid residues. The T1R3 protein contains 855 amino acid residues.

Transmembrane regions. The number of membrane spanning domains for pig T1R1 and T1R3 was determined using several bioinformatic programs. The translated amino acid sequence for pig T1R1 and T1R3 was used in these analyses. The programs used included TMHMM server 2 (on the worldwide web at cbs.dtu.dk/services/TMHMM-2.0/), SOSUI version 1.11 (on the worldwide web at bp.nuap.nagoya-u.ac.jp/sosui/sosui_submit.html), HMMTOP (on the worldwide web at enzim.hu/hmmtop/html/submit.html) and TMpred (on the worldwide web at ch.embnet.org/software/TMPRED_form.html).

Mouse T1R1 (GenBank acc. # EDL14920) and T1R3 (GenBank acc. # EDL15046) amino acid sequences were used to evaluate these programs since the number and location of their transmembrane regions have been reported. Based upon these results, the TMHMM server 2 program was found to provide results that were most consistent with published reports.

### Results

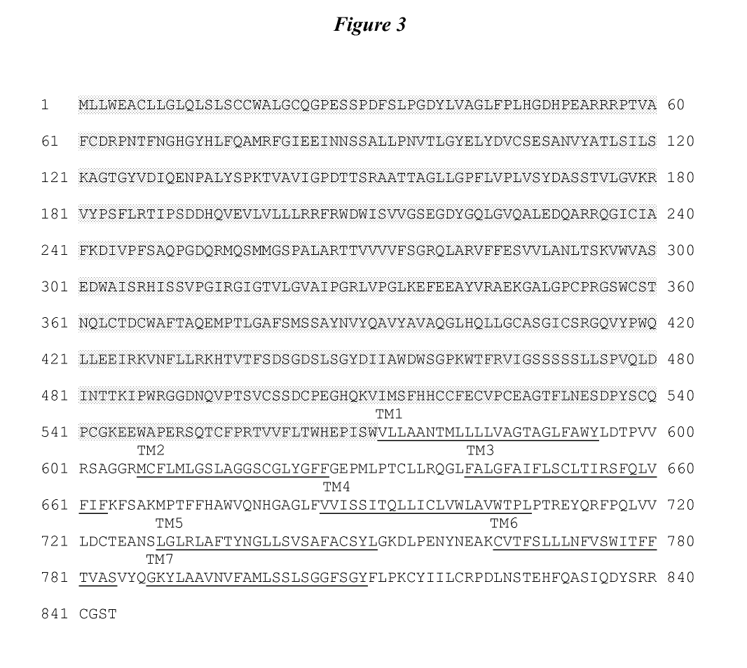

Analysis of pig T1R1 with the TMHMM server 2 program resulted in 7 transmembrane regions (Table 3 and FIG. 3) that were similar in number and location to mouse T1R1.

However, while 7 transmembrane domains were identified for mouse T1R3, the predicted location of the first transmembrane region was located between amino acids 4-26; however, this is not consistent with published reports for mouse T1R3. A membrane spanning domain at this location would result in the ligand binding domain being located within the cytosol of cell. Such a location would make the receptor inaccessible to the ligand. Moreover, when the pig T1R3 sequence was analyzed with the TMHMM server 2, only six transmembrane regions were identified. These regions were similar in location to those predicted for mouse T1R3, with the exception of a membrane spanning domain located between amino acids 4-26. It appears that the TMHMM program provides erroneous results with the analysis of the T1R3 sequences in mouse and pig, so an alternative approach was utilized.

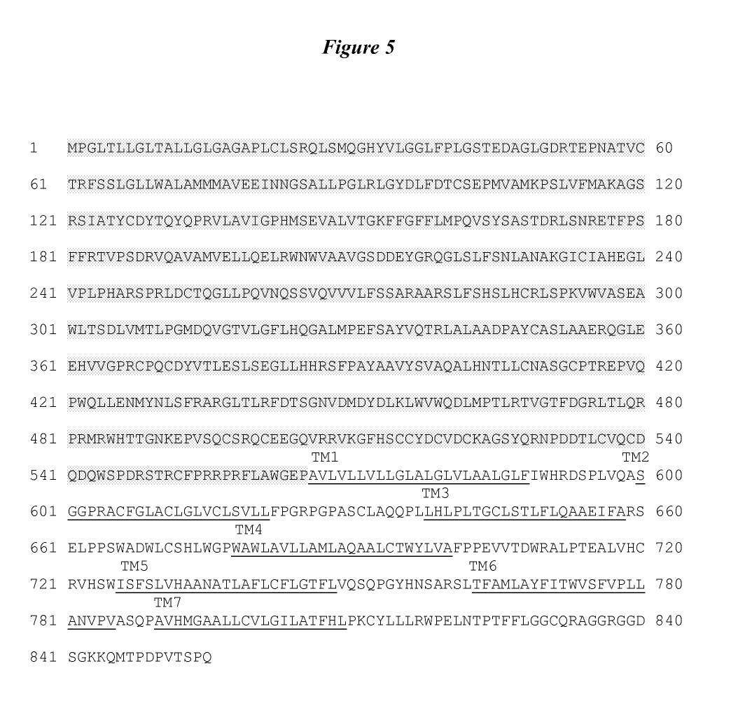

Human, mouse and pig T1R3 amino sequences were aligned using ClustalW to allow for visual inspection and analysis of membrane spanning domains (FIG. 4). The membrane spanning domains for human and mouse T1R3 primary amino acid sequences were based upon UniProtKB/Swiss-Prot entries Q7RTX0 and Q91VA4, respectively. The location and identity of TM regions identified for pig T1R3 using TMHMM were compared to those of human and mouse T1R3. The location and number of TM regions in human and mouse T1R3 were similar to those predicted within the pig T1R3 sequence, with the exception of one. The region corresponding to TM3 in human and mouse T1R3 did not correspond to a similar membrane spanning domain in pig T1R3. In this stretch of 21 amino acid residues, the pig T1R3 amino acid sequence is 85.7% identical to mouse T1R3 and 90.5% identical to human T1R3. The differences between amino acids is unlikely to have any conformational implications based upon the properties of their functional side changes. Therefore, based upon alignment of T1R3 amino acid sequences and the high degree of conservation of amino acid residues within the human and mouse T1R3 TM3 region, the location of this membrane spanning domain is likely conserved within the pig T1R3 protein as well. Consequently, the pig T1R3 protein is predicted to contain seven transmembrane regions that are similar in number and location to human and mouse T1R3 proteins. The number and location of pig T1R3 transmembrane domains is shown in Table 3 and FIG. 5.

Amino acid residues. The number and location of serine, threonine and tyrosine residues involved in phosphorylation were identified in pig T1R1 and T1R3 amino acid sequence using the NetPhos 2.0 server (on the worldwide web at cbs.dtu.dk/services/NetPhos/) and are shown in Table 3.

Post-translational modification and subcellular localization. The presence of a signal peptide involved in the targeting of pig T1R1 and T1R3 proteins within the cell was determined using the to modified post-translationally was determined using Signal P 3.0 server (on the worldwide web at cbs.dtu.dk/services/SignalP/). N-glycosylation sites were identified using the NetNGlyc 1.0 server (on the worldwide web at cbs.dtu.dk/services/NetNGlyc/). The endoplasmic reticulum retention signal, peroxisomal target sequence, actin-binding motif, N-myristoylation motif, transport motif from cell surface to golgi, tyrosines at C-terminus, dileucine motif at C-terminus and subcellular localization was determine using the PSORT II Prediction software (on the worldwide web at psort.ims.u-tokyo.ac.j.p/form2.html).

TABLE 3
Identification of functional domains and amino acid
residues of importance in pig T1R1 and T1R3 proteins.

| Amino acid regions/residues | Pig T1R1 | Pig T1R3 |
|---|---|---|
| Ligand binding domain | 1-571 | 1-565 |
| Signal peptide | None | 1-18 |
| N-glycosylation sites | None | 56 |
| | | 83 |
| | | 262 |
| | | 409 |
| | | 430 |
| | | 735 |
| Serine phosphorylation sites | 28 | 28 |
| | 173 | 43 |
| | 184 | 166 |
| | 216 | 173 |
| | 248 | 180 |
| | 258 | 187 |
| | 310 | 248 |
| | 356 | 283 |
| | 382 | 291 |
| | 445 | 378 |
| | 447 | 526 |
| | 469 | 545 |
| | 472 | 549 |
| | 475 | 665 |
| | 498 | 724 |
| | 502 | 841 |
| | 666 | |
| | 804 | |
| | 826 | |
| | 833 | |
| | 838 | |
| Threonine phosphorylation sites | 58 | 100 |
| | 124 | 125 |
| | 148 | 169 |
| | 149 | 303 |
| | 154 | 439 |
| | 155 | 444 |
| | 269 | 465 |
| | 320 | 471 |
| | 554 | 477 |
| | 597 | 488 |
| | 653 | 534 |
| | 705 | 847 |
| Tyrosine phosphorylation sites | 113 | 216 |
| | 126 | 335 |
| | 220 | 348 |
| | 341 | 373 |
| | 449 | 392 |
| | 537 | 527 |
| | 623 | 755 |
| Transmembrane regions | 7 | 7 |
| | TM1(572-594) | TM1(566-588) |
| | TM2(607-626) | TM2(600-622) |
| | TM3(641-663) | TM3(641-663)* |
| | TM4(683-705) | TM4(678-700) |
| | TM5(729-751) | TM5(726-748) |
| | TM6(764-786) | TM6(763-785) |
| | TM7(790-812) | TM7(790-809) |
| N-terminus location | Extracellular | Extracellular |
| C-terminus location | Intracellular | Intracellular |
| Endoplasmic reticulum retention | None | None |
| signal | | |
| Peroxisomal target sequence | None | None |
| Actin-binding motif | None | None |
| N-myristoylation motif | None | None |
| Transport motif from cell surface | None | None |
| to golgi | | |
| Tyrosines at C-terminus | None | None |
| Dileucine motif at C-terminus | None | None |
| Subcellular localization | Plasma membrane | Plasma membrane |

*This transmembrane region was predicted based upon ClustalW alignment of human, mouse and pig T1R3 amino acid sequence.

It is understood that the examples and embodiments described herein are for illustrative purposes only and that various modifications or changes in light thereof will be suggested to persons skilled in the art and are to be included within the spirit and purview of this application and scope of the appended claims. All publications, accession numbers, patents, and patent applications cited herein are hereby incorporated by reference in their entirety for all purposes.

INFORMAL SEQUENCE LISTING

SEQ ID NO: 1
Pig TAS1R1
ATGTTGCTCTGGGAGGCGTGCCTGCTGGGTCTGCAGCTCTCCCTCTCCTGCTGCTGGGCTTTGGGCTGCC
AGGGCCCAGAGTCCTCCCCTGACTTCAGCCTCCCTGGGGACTACCTTGTAGCTGGCCTCTTCCCTCTACA
CGGTGACCACCCAGAGGCGAGGCGCAGACCCACAGTGGCCTTCTGTGACAGACCCAACACCTTCAACGGC
CACGGCTACCACCTCTTCCAAGCCATGCGGTTTGGCATCGAGGAGATAAACAACTCCAGCGCCCTGCTGC
CCAACGTCACTCTGGGGTATGAGCTATACGACGTGTGCTCGGAGTCCGCGAACGTGTACGCCACGCTGAG
CATCCTGAGCAAGGCTGGGACAGGCTACGTGGACATCCAAGAAAACCCTGCCCTCTATTCCCCCAAGACC
GTGGCGGTCATCGGGCCCGACACCACCAGCCGCGCTGCCACCACCGCAGGCCTGCTGGGCCCCTTCCTGG
TGCCCCTGGTCAGCTATGATGCCAGCAGCACGGTGCTGGGTGTGAAGCGGGTGTACCCCTCTTTCCTGCG
CACCATCCCCAGCGATGACCACCAGGTGGAGGTCTTGGTGCTGCTGCTGCGGAGGTTCAGGTGGGACTGG
ATCTCGGTGGTGGGCAGCGAAGGTGACTACGGGCAGCTGGGGGTGCAGGCGCTGGAGGACCAGGCCCGCC
GGCAGGGCATCTGCATTGCCTTCAAGGACATCGTGCCGTTCTCTGCCCAGCCGGGCGACCAGAGGATGCA
GAGCATGATGGGCAGCCCGGCTCTCGCAAGGACCACCGTTGTGGTCGTTTTCTCCGGTCGGCAGCTGGCC
AGGGTGTTCTTTGAGTCTGTGGTGCTGGCCAACCTGACCTCCAAGGTGTGGGTCGCCTCCGAGGACTGGG
CCATCTCCAGACACATCAGCAGTGTGCCCGGGATCCGGGGCATCGGCACAGTGCTGGGCGTGGCCATCCC
CGGGAGGCTGGTGCCCGGCCTGAAGGAGTTTGAGGAGGCCTACGTCAGGGCAGAGAAAGGGGCCCTTGGG
CCTTGCCCCAGGGGCTCCTGGTGCAGCACCAACCAGCTCTGCACAGACTGCTGGGCTTTCACGGCCCAGG
AGATGCCCACACTTGGAGCATTCTCCATGAGTTCTGCCTACAACGTGTACCAGGCTGTCTATGCCGTGGC
CCAGGGCCTCCACCAGCTCCTGGGCTGTGCCTCTGGCATCTGTTCTCGAGGCCAAGTCTACCCCTGGCAG
CTCCTGGAGGAGATCCGCAAGGTCAATTTCCTTCTACGCAAGCACACTGTGACATTTAGTGACAGTGGGG
ACTCGCTCAGCGGCTATGACATAATTGCCTGGGACTGGAGTGGCCCCAAGTGGACCTTCAGGGTCATCGG
CTCGTCATCGTCGTCATTGTTGTCGCCAGTTCAGCTAGACATAAATACTACCAAAATCCCATGGCGTGGC
GGGGACAACCAGGTGCCGACGTCCGTGTGTTCCAGCGACTGTCCTGAGGGCCACCAGAAAGTGATCATGA
GTTTCCACCACTGCTGCTTTGAGTGTGTGCCTTGTGAGGCCGGGACCTTCCTCAACGAGAGTGACCCCTA
CAGCTGCCAACCCTGTGGGAAAGAAGAGTGGGCACCTGAGAGAAGCCAGACTTGCTTCCCACGCACCGTG
GTATTCTTGACTTGGCATGAGCCCATCTCTTGGGTGCTGCTAGCAGCTAACACGATGTTGCTGCTGCTGG
TGGCTGGGACTGCTGGCCTGTTTGCTTGGTACCTAGACACCCCTGTGGTGAGGTCCGCTGGGGGCAGAAT
GTGCTTCCTCATGCTGGGCTCCCTGGCAGGGGGCAGCTGCGGCCTCTATGGCTTTTTTGGGGAGCCCATG
CTGCCCACATGCTTGCTGCGCCAAGGCCTCTTTGCCCTTGGTTTTGCCATCTTCCTGTCCTGCCTGACAA
TCCGCTCTTTCCAACTTGTCTTCATCTTCAAGTTTTCTGCCAAGATGCCCACATTCTTTCACGCGTGGGT
CCAAAACCATGGTGCTGGCCTGTTTGTGGTGATCAGCTCGATAACCCAGCTGCTTATCTGTCTGGTTTGG
CTGGCGGTGTGGACCCCACTGCCCACCAGGGAATACCAGCGCTTCCCACAGCTGGTGGTGCTTGACTGCA
CAGAAGCGAATTCACTGGGCTTAAGGCTGGCTTTCACCTACAACGGTCTCCTCTCGGTCAGCGCCTTTGC
CTGCAGCTACCTGGGTAAGGACCTGCCAGAGAACTACAATGAGGCCAAATGTGTTACCTTCAGCCTGCTC
CTCAACTTCGTGTCCTGGATCACCTTCTTCACCGTGGCCAGTGTCTACCAGGGCAAGTACCTGGCCGCCG
TCAACGTGTTTGCCATGCTGAGCAGCCTGAGTGGAGGCTTCAGCGGTTATTTCCTCCCCAAATGCTATAT
CATCCTGTGCCGCCCAGATCTCAACAGCACGGAGCACTTCCAGGCCTCCATCCAGGACTACAGCAGGCGC
TGCGGCTCCACCTGA
SEQ ID NO: 2
Pig TAS1R3
ATGCCAGGCCTGACTCTCCTGGGCCTCACAGCTCTCCTGGGCCTCGGGGCGGGGGCCCCGCTGTGCCTGT
CCCGGCAGCTCAGCATGCAAGGGCACTACGTGCTGGGGGGGCTCTTCCCCCTGGGCTCCACCGAGGATGC
TGGGCTGGGCGACAGGACAGAGCCCAACGCCACCGTGTGTACCAGGTTCTCGTCCCTCGGCCTGCTCTGG
GCACTGGCCATGATGATGGCTGTGGAGGAAATCAACAACGGATCGGCCCTGCTCCCTGGGCTGCGCCTGG
GCTACGACCTCTTCGACACCTGCTCAGAGCCCATGGTCGCCATGAAGCCCAGCCTAGTGTTCATGGCCAA
GGCTGGCAGCCGCAGCATTGCCACCTACTGCGACTACACGCAGTACCAGCCCCGCGTGCTGGCCGTCATC
GGGCCCCACATGTCTGAGGTCGCCCTGGTCACCGGCAAGTTCTTCGGCTTCTTCCTCATGCCGCAGGTCA
GCTACAGCGCCAGCACGGACCGGCTGAGCAACCGCGAGACGTTCCCCTCCTTCTTCCGCACGGTGCCCAG
CGACCGCGTGCAGGCAGTGGCCATGGTGGAGCTGCTGCAGGAGCTGCGTTGGAACTGGGTGGCCGCCGTG
GGCAGCGACGACGAGTACGGCCGGCAGGGCCTGAGCCTCTTCTCCAACCTGGCCAACGCCAAGGGCATCT
GCATCGCGCACGAGGGCCTGGTGCCGCTGCCCCACGCCCGTAGCCCAAGGCTGGACTGCACGCAGGGCCT
GCTGCCCCAAGTGAACCAGAGCAGCGTGCAGGTAGTGGTGCTGTTCTCCTCCGCCCGCGCCGCCCGCAGC
CTCTTCAGCCACAGCCTCCACTGCAGGCTCTCGCCCAAGGTGTGGGTGGCCAGCGAGGCCTGGCTGACCT
CGGACCTGGTCATGACACTGCCGGGCATGGACCAGGTGGGCACCGTGCTCGGCTTCCTGCACCAGGGTGC
CCTGATGCCCGAGTTCTCGGCCTACGTGCAGACCCGCCTGGCCCTGGCCGCCGACCCCGCCTACTGCGCC
TCGCTGGCTGCCGAGCGGCAGGGGCTGGAGGAGCACGTGGTGGGGCCTCGCTGCCCCCAGTGTGACTACG
TCACATTGGAGAGCCTGTCCGAGGGGCTGCTGCACCACCGGAGCTTCCCCGCCTACGCGGCTGTGTACAG
CGTGGCCCAGGCGCTCCACAACACGCTGCTCTGCAACGCCTCGGGCTGCCCCACGCGGGAGCCGGTGCAG
CCCTGGCAGCTCCTGGAGAACATGTACAACCTGAGCTTCCGTGCGCGCGGCCTGACACTGCGGTTCGACA
CCAGCGGGAACGTGGATATGGATTACGACCTGAAGCTGTGGGTGTGGCAGGACCTGATGCCCACACTGCG
CACCGTGGGCACTTTCGACGGCCGCCTGACGCTCCAGCGTCCCCGGATGCGCTGGCACACAACGGGGAAC
AAGGAGCCCGTGTCCCAGTGCTCTCGACAGTGCGAGGAGGGCCAGGTGCGCCGGGTGAAGGGCTTCCACT
CCTGCTGCTATGACTGTGTGGACTGCAAGGCGGGCAGCTACCAGCGCAACCCAGATGACACCCTCTGTGT
CCAGTGTGACCAGGACCAGTGGTCCCCGGACCGGAGCACCCGGTGTTTCCCCCGCAGGCCCAGGTTCCTG
GCTTGGGGGGAGCCAGCCGTGCTGGTGCTGCTCGTGCTGCTGGGCCTGGCTCTGGGCCTGGTGCTGGCAG
CCCTGGGGCTCTTCATCTGGCACCGAGACAGCCCACTGGTTCAGGCCTCCGGTGGGCCACGGGCCTGCTT
CGGCTTGGCCTGCCTGGGCCTCGTCTGCCTGAGCGTCCTCCTGTTCCCGGGCCGGCCCGGCCCTGCCAGC
TGCCTGGCCCAACAGCCGCTGCTCCACCTCCCTCTCACTGGCTGCCTGAGCACCCTCTTCCTGCAGGCGG
CTGAGATCTTTGCGAGGTCAGAGCTGCCGCCAAGCTGGGCCGACTGGCTCTGTAGCCACCTGTGGGGGCC
CTGGGCCTGGCTGGCGGTGCTGCTCGCCATGCTGGCACAAGCAGCGCTCTGCACCTGGTACCTGGTGGCC
TTCCCACCGGAGGTGGTGACAGACTGGCGTGCGCTGCCCACAGAGGCACTGGTGCACTGCCGCGTGCACT
CCTGGATCAGCTTCAGCCTGGTACATGCTGCCAATGCCACGCTGGCTTTCCTCTGCTTCCTGGGCACCTT
CCTGGTGCAGAGCCAGCCTGGCTACCACAACAGCGCCCGCAGCCTGACCTTCGCCATGCTGGCCTACTTC
ATCACCTGGGTTTCCTTTGTGCCCCTCCTAGCCAACGTGCCTGTGGCCTCCCAGCCTGCCGTGCACATGG
GTGCCGCCCTCCTCTGTGTGCTGGGCATCCTGGCCACTTTCCACCTGCCCAAGTGCTACCTGCTGCTGAG
GTGGCCAGAGCTCAACACCCCCACGTTCTTCCTGGGGGGATGCCAGAGGGCAGGGGGGCGGGGAGGAGAC
TCTGGGAAAAAACAAATGACCCCTGACCCAGTGACCTCCCCCCAGTGA
SEQ ID NO: 3
T1R3 GSP forward

| | |
|---|---|
| 5′-ACTGCCGCGTGCACTCCTG-3′ | (SEQ ID NO: 3) |

SEQ ID NO: 4
T1R3 GSP reverse

| | |
|---|---|
| 5′-GGAGGCCACAGGCACGTTG-3′ | (SEQ ID NO: 4) |

SEQ ID NO: 5
T1R1 DP forward

| | |
|---|---|
| 5′-GGCAGTACCCCTCCTTCCTGMGNACNATHCC-3′ | (SEQ ID NO: 5) |

SEQ ID NO: 6
T1R1 DP reverse

| | |
|---|---|
| 5′-CCTGCACGCCCAGCTGNCCRTARTC-3' | (SEQ ID NO: 6) |

SEQ ID NO: 7
T1R3 forward

| | |
|---|---|
| 5'-ATGCCAGGCCTGACTCTCCTG-3′ | (SEQ ID NO: 7) |

SEQ ID NO: 8
T1R3 reverse

| | |
|---|---|
| 5′-TCACTGTGGGGTCAAGGGTGG-3′ | (SEQ ID NO: 8) |

SEQ ID NO: 9
T1R3 nested reverse

| | |
|---|---|
| 5′-TCAGGGCACAGCCTGCTGGT-3′ | (SEQ ID NO: 9) |

SEQ ID NO: 10
T1R1 forward

| | |
|---|---|
| 5′-ATGCCAGGCTCAGGGCGG-3′ | (SEQ ID NO: 10) |

SEQ ID NO: 11
T1R1 nested forward

| | |
|---|---|
| 5′-ATGTTGCTCTGGGAGGCGTG-3′ | (SEQ ID NO: 11) |

SEQ ID NO: 12
T1R1 reverse

| | |
|---|---|
| 5'-TCAGGTGGAGCCGCAGCG-3' | (SEQ ID NO: 12) |

SEQ ID NO: 13
Pig T1R1
MLLWEACLLGLQLSLSCCWALGCQGPESSPDFSLPGDYLVAGLFPLHGDHPEARRRPTVA
FCDRPNTFNGHGYHLFQAMRFGIEEINNSSALLPNVTLGYELYDVCSESANVYATLSILS
KAGTGYVDIQENPALYSPKTVAVIGPDTTSRAATTAGLLGPFLVPLVSYDASSTVLGVKR
VYPSFLRTIPSDDHQVEVLVLLLRRFRWDWISVVGSEGDYGQLGVQALEDQARRQGICIA
FKDIVPFSAQPGDQRMQSMMGSPALARTTVVVVFSGRQLARVFFESVVLANLTSKVWVAS
EDWAISRHISSVPGIRGIGTVLGVAIPGRLVPGLKEFEEAYVRAEKGALGPCPRGSWCST
NQLCTDCWAFTAQEMPTLGAFSMSSAYNVYQAVYAVAQGLHQLLGCASGICSRGQVYPWQ
LLEEIRKVNFLLRKHTVTFSDSGDSLSGYDIIAWDWSGPKWTFRVIGSSSSSLLSPVQLD
INTTKIPWRGGDNQVPTSVCSSDCPEGHQKVIMSFHHCCFECVPCEAGTFLNESDPYSCQ
PCGKEEWAPERSQTCFPRTVVFLTWHEPISWVLLAANTMLLLLVAGTAGLFAWYLDTPVV
RSAGGRMCFLMLGSLAGGSCGLYGFFGEPMLPTCLLRQGLFALGFAIFLSCLTIRSFQLV
FIFKFSAKMPTFFHAWVQNHGAGLFVVISSITQLLICLVWLAVWTPLPTREYQRFPQLVV
LDCTEANSLGLRLAFTYNGLLSVSAFACSYLGKDLPENYNEAKCVTFSLLLNFVSWITFF
TVASVYQGKYLAAVNVFAMLSSLSGGFSGYFLPKCYIILCRPDLNSTEHFQASIQDYSRR
CGST
SEQ ID NO: 14
Pig T1R3
MPGLTLLGLTALLGLGAGAPLCLSRQLSMQGHYVLGGLFPLGSTEDAGLGDRTEPNATVC
TRFSSLGLLWALAMMMAVEEINNGSALLPGLRLGYDLFDTCSEPMVAMKPSLVFMAKAGS
RSIATYCDYTQYQPRVLAVIGPHMSEVALVTGKFFGFFLMPQVSYSASTDRLSNRETFPS
FFRTVPSDRVQAVAMVELLQELRWNWVAAVGSDDEYGRQGLSLFSNLANAKGICIAHEGL
VPLPHARSPRLDCTQGLLPQVNQSSVQVVVLFSSARAARSLFSHSLHCRLSPKVWVASEA
WLTSDLVMTLPGMDQVGTVLGFLHQGALMPEFSAYVQTRLALAADPAYCASLAAERQGLE
EHVVGPRCPQCDYVTLESLSEGLLHHRSFPAYAAVYSVAQALHNTLLCNASGCPTREPVQ
PWQLLENMYNLSFRARGLTLRFDTSGNVDMDYDLKLWVWQDLMPTLRTVGTFDGRLTLQR
PRMRWHTTGNKEPVSQCSRQCEEGQVRRVKGFHSCCYDCVDCKAGSYQRNPDDTLCVQCD
QDQWSPDRSTRCFPRRPRFLAWGEPAVLVLLVLLGLALGLVLAALGLFIWHRDSPLVQAS
GGPRACFGLACLGLVCLSVLLFPGRPGPASCLAQQPLLHLPLTGCLSTLFLQAAEIFARS
ELPPSWADWLCSHLWGPWAWLAVLLAMLAQAALCTWYLVAFPPEVVTDWRALPTEALVHC
RVHSWISFSLVHAANATLAFLCFLGTFLVQSQPGYHNSARSLTFAMLAYFITWVSFVPLL
ANVPVASQPAVHMGAALLCVLGILATFHLPKCYLLLRWPELNTPTFFLGGCQRAGGRGGD
SGKKQMTPDPVTSPQ

What is claimed is:

1. A method of identifying a compound that modulates signal transduction of a porcine taste receptor in taste cells, the method comprising the steps of
(i) contacting the compound with a porcine taste receptor comprising: a pT1R1 polypeptide having at least 90% sequence identity to SEQ ID NO:1; and a pT1R3 polypeptide having at least 90% sequence identity to SEQ ID NO:2; and
(ii) determining a physical or functional effect of the compound upon the receptor, thereby identifying a compound that modulates porcine taste receptor signal transduction.

2. The method of claim 1, wherein the pT1R1 polypeptide has at least 95% sequence identity to SEQ ID NO:1; and the pT1R3 polypeptide has at least 95% sequence identity to SEQ ID NO:2.

3. The method of claim 1, wherein the pT1R1 polypeptide has the amino acid sequence of SEQ ID NO:1; and the pT1R3 polypeptide has the amino acid sequence of SEQ ID NO:2.

4. The method of claim 1, wherein the receptor has G protein coupled receptor activity.

5. The method of claim 1, wherein the functional effect is receptor-mediated calcium flux.

6. The method of claim 1, wherein the physical effect is binding of the compound to the receptor.

7. The method of claim 1, wherein the receptor is expressed in a cell that does not natively express pT1R1 or pT1R3.

8. The method of claim 1, wherein the compound increases porcine taste receptor signal transduction.

9. The method of claim 8, further comprising the step of determining the palatability of the compound to a porcine.

10. The method of claim 8, further comprising selecting the compound as a supplement for porcine feed.

11. The method of claim 1, wherein the compound is an amino acid.

12. The method of claim 1, wherein the compound is a peptide.

13. The method of claim 1, wherein the compound is a dipeptide.

14. A porcine foodstuff or medicine supplemented with a compound selected according to the method of claim 1.
